(19) United States
(12) Patent Application Publication
(10) Pub. No.: US 2012/0303082 A1
(43) Pub. Date: Nov. 29, 2012

(54) Adjusting Cardiac Pacing Response Sensing Intervals

(76) Inventors: Yanting Dong, Lexington, KY (US); Shibaji Shome, Arden Hills, MN (US); Aaron McCabe, Minneapolis, MN (US); Amy J. Brisben, St. Paul, MN (US); Clayton Foster, Andover, MN (US); David W. Yost, Brooklyn Park, MN (US); Kenneth N. Hayes, Blaine, MN (US)

(21) Appl. No.: 13/306,611

(22) Filed: Nov. 29, 2011

Related U.S. Application Data
(60) Provisional application No. 61/419,140, filed on Dec. 2, 2010.

(51) Int. Cl. A61N 1/37; A61N 1/365

(52) U.S. Cl. 607/17; 607/28

(57) ABSTRACT

Discrimination between different types of possible cardiac pacing responses may depend on the timing of expected features that are sensed within a temporal framework. The temporal framework may include classification intervals, blanking periods and appropriately timed back up paces. The classification intervals and blanking periods of the temporal framework are intervals of time that have time parameters that include start time, end time, and length. The relationships and timing parameters of the elements of the temporal framework, e.g., blanking periods, classification intervals, delay periods, and backup pacing, should support detection of features used to discriminate between different types of pacing responses. As the system learns the morphology of the particular patient by analyzing the waveform of the pacing response signal, the temporal framework for pacing response determination may be adjusted to accommodate the individual patient.

### RELATED PATENT DOCUMENTS

This application claims the benefit of Provisional Patent Application Ser. No. 61/419,140, filed on Dec. 2, 2010, to which priority is claimed pursuant to 35 U.S.C. §119(e) and which is hereby incorporated herein by reference.

### FIELD OF THE INVENTION

The present invention relates generally to implantable medical devices and, more particularly, to cardiac pacing response classification.

### BACKGROUND

Cardiac pacing devices operate to stimulate the heart tissue electrically coupled to the electrodes to produce a contraction of the tissue. Pacemakers deliver a series of low energy pace pulses timed to assist the heart in producing a contractile rhythm that maintains cardiac pumping efficiency. Pace pulses may be intermittent or continuous, depending on the needs of the patient. There exist a number of categories of cardiac devices that provide pacing pulses, with various modes for sensing and pacing one or more heart chambers.

When a pace pulse produces a contraction in the heart tissue, the electrical cardiac signal following the contraction is denoted the evoked response signal. A pace pulse must exceed a minimum energy value, or capture threshold, to produce a contraction. It is desirable for a pace pulse to have sufficient energy to stimulate capture of the heart without expending energy significantly in excess of the capture threshold. Thus, accurate determination of the capture threshold may be required for efficient pace energy management. If the pace pulse energy is too low, the pace pulses may not reliably produce a contractile response in the heart and may result in ineffective pacing. If the pace pulse energy is too high, the patient may experience discomfort and/or the battery life of the device will be shorter.

Capture detection allows the cardiac device to adjust the energy level of pace pulses to correspond to the optimum energy expenditure that reliably produces a contraction. Further, capture detection allows the cardiac device to initiate a back-up pulse whenever a pace pulse does not produce a contraction.

### SUMMARY

Embodiments described herein involve methods of operating a cardiac device. Pacing pulses are delivered to a heart chamber during a cardiac cycle. A cardiac pacing response signal of the heart chamber is sensed during the cardiac cycle and following the pacing pulse in one or both of a first classification interval and a second classification interval, each of the first and second classification intervals associated with one or more timing parameters including at least a start time. At least one timing parameter of the first classification interval, the second classification interval, and one or more blanking periods is adapted based on timing of at least one signal feature of the pacing response signal and a temporal relationship between the first classification interval and the second classification interval. In some embodiments the signal feature comprises a positive or negative peak. The first and second classification intervals, having the adapted timing parameters to a subsequent pacing response signal sensed following a subsequent pacing pulse delivered to the heart chamber, are applied. It is determined whether the signal feature of the subsequent pacing response signal falls within the first or second classification intervals that have the same timing parameters. A pacing response of the heart chamber to the to the subsequent cardiac pacing pulse based on a determination that the signal feature falls within the first or second classification intervals having the adapted timing parameters is classified. Cardiac therapy is delivered based on the classification of the pacing response.

In some implementations adapting the timing parameters based on the temporal relationship of the first and second classification intervals comprises adapting a start time of the second classification interval based on an end time of the first classification interval. In other implementations adapting the timing parameter comprises adapting a timing parameter of a blanking period based on a temporal relationship between the blanking period and one or both of the first classification interval and the second classification interval. In yet another implementation adapting the timing parameter comprises adapting timing parameters of three classification intervals, the first classification interval used to detect possible capture or fusion, the second classification interval used to confirm fusion, and a third classification interval used to confirm capture. In some embodiments adapting the timing parameter comprises at least one of shortening one or more of the blanking periods and lengthening one or more of the first and second classification intervals and lengthening one or more of the blanking periods and shortening one or more of the first and second classification intervals.

According to some embodiments an amount of change in the timing of the signal feature is compared to an initial timing of the signal feature to a threshold and a determination is made whether to adapt one or both of the first and second classification intervals based on the comparison of the amount of change. Some implementations may also include comparing an amount of change in the timing of the signal feature compared to an initial timing of the signal feature to a threshold and determining whether to re-initialize timing parameters of the first classification interval, the second classification interval, and the one or more blanking periods, wherein re-initializing the timing parameters involves acquiring a multi-sample electrogram of a pacing response signal.

Embodiments described herein include a device comprising pacing circuitry configured to deliver a pacing pulse to a heart chamber during a cardiac cycle. The device also includes sensing circuitry configured to sense a cardiac pacing response signal of the heart chamber during the cardiac cycle and following the pacing pulse in one or both of a first classification interval and a second classification interval, each of the first and second classification intervals associated with one or more timing parameters including at least a start time. Additionally, the device includes control circuitry configured to adapt at least one timing parameter of one or more of the first classification interval, the second classification interval, and one or more blanking periods based on timing of at least one signal feature of the pacing response signal and a temporal relationship between the first classification interval and the second classification interval. The control circuitry is also configured to apply the first and second classification intervals having the adapted timing parameters to a subsequent pacing response signal sensed following a subsequent pacing pulse delivered to the heart chamber and to determine if the signal feature of the subsequent pacing response signal falls within the first or second classification intervals that have the adapted timing parameters. The control circuitry may also be configured to classify a pacing response of the heart chamber to the subsequent cardiac pacing pulse based on a determination that the signal feature falls within the first or second classification intervals having the adapted timing parameters and to deliver cardiac therapy based on classification of the pacing response.

Some embodiments may include that the control circuitry is further configured to adapt the timing parameters based on the temporal relationship of the first and second classification intervals comprises adapting a start time of the second classification interval based on an end time of the first classification interval. In some implementations that control circuitry may also be configured to adapt a timing parameter of a blanking period based on a temporal relationship between the blanking period and one or both of the first classification interval and the second classification interval. Additionally, in some embodiments, the control circuitry my be further configured to adapt timing parameters of three classification intervals, the first classification interval used to detect possible capture or fusion, the second classification interval used to confirm fusion, and a third classification interval used to confirm capture. Some implementations may also include that the control circuitry is further configured to compare an amount of change in the timing of the signal feature compared to an initial timing of the signal feature to a threshold and to determine whether to adapt one or both of the first and second classification intervals based on the comparison of the amount of change.

Some embodiments for operating a cardiac device include delivering at least one pacing pulse to a heart chamber and sensing a pacing response signal of the heart chamber following the pacing pulse. The embodiments may also include detecting a temporal event of the pacing response signal, the temporal event comprising a point in time that falls between a first feature and a second feature of the pacing response signal. Additionally, in some embodiments of operating a cardiac device include initializing timing parameters of one or more of pacing response classification intervals and one or more blanking periods based on the detected temporal event so that the first feature falls within a first classification interval and the second feature falls within a second classification interval.

Some implementations may include that sensing the pacing response further comprises acquiring a multi-sample of the pacing response signal. In other embodiments may include that wherein detecting the temporal event comprises detecting a zero crossing point of the pacing response signal. In other implementations detecting the temporal event comprises detecting an inflection point of the pacing response signal. In yet other implementations detecting the temporal event comprises detecting a midpoint between a time of occurrence of the first feature and a time of occurrence of the second feature. Some embodiments further include detecting the temporal event comprises detecting a zero crossing point or an inflection point of the cardiac signal initializing the timing parameters of the one or more classification intervals and the one or more blanking periods comprises setting a start time of a blanking period and an end time of a classification interval to coincide with the zero crossing point or inflection point. Some embodiments may further include initializing the timing parameters of the one or more blanking periods to allow sensing of the first or second feature and/or initializing the timing parameters of the one or more blanking periods to prevent sensing of signal features other than the first or second features

Embodiments described herein include a cardiac device comprising pacing circuitry configured to deliver at least one pacing pulse to a heart chamber. In this case, the cardiac device also includes sensing circuitry configured to sense a pacing response signal of the heart chamber following the pacing pulse and detect a temporal event of the pacing response signal, the temporal event comprising a point in time that falls between a first feature and a second feature of the pacing response signal. The cardiac device may also include control circuitry configured to initialize timing parameters of one or more of pacing response classification intervals and one or more blanking periods based on the detected temporal event so that the first feature falls within a first classification interval and the second feature falls within a second classification interval.

Some implementations described herein include that the sensing circuitry is further configured to acquire a multi-sample electrogram of the response signal. Additional embodiments include that the sensing circuitry is further configured to detect a zero crossing point or an inflection point of the cardiac signal and that the control circuitry is further configured to initialize the timing parameters of the one or more classification intervals and the one or more blanking periods and to set a start time of a blanking period and an end time of a classification interval to coincide with the zero crossing point or inflection point.

For some implementation a method includes delivering a pacing pulse to a left ventricle of a heart and sensing a cardiac pacing response signal of the left ventricle. The method also includes detecting a first peak in a first capture detection interval and detecting a second peak in one of a fusion detection interval and a second capture detection interval that follows the fusion detection interval. Additionally the method comprises discriminating between capture and fusion based on the first and second peaks.

In some implementations detecting the first peak in the first capture detection interval comprises detecting the first peak in a capture detection region within the first capture detection interval, the capture detection region having upper and lower timing boundaries and upper and lower amplitude boundaries. In other implementations, discriminating between capture and fusion comprises classifying the pacing response as potential capture if the first peak falls within the capture detection region and confirming capture if the second peak falls within the second capture detection interval. In additional embodiments discriminating between capture and fusion comprises classifying the pacing response as fusion if the first peak falls does not within the capture detection region or confirming fusion if the second peak falls within the fusion detection interval.

Implementations described herein include a device comprising pacing circuitry configured to deliver a pacing pulse to a left ventricle of a heart. The device also includes sensing circuitry configured to sense a cardiac pacing response signal of the left ventricle and detect a first peak in a first capture detection interval and detecting a second peak in one of a fusion detection interval and a second capture detection interval that follows the fusion detection interval. Additionally, the device includes control circuitry configured to discriminate between capture and fusion based on the first and second peaks. In some implementations the sensing circuitry is configured to detect the first peak in a capture detection region within the first capture detection interval, the capture detection region having upper and lower timing boundaries and upper and lower amplitude boundaries.

The above summary is not intended to describe each embodiment or every implementation of the present invention. Advantages and attainments, together with a more complete understanding of the invention, will become apparent and appreciated by referring to the following detailed description and claims in conjunction with the accompanying drawings.

### BRIEF DESCRIPTION OF THE DRAWINGS

FIG. 1 illustrates several types of cardiac pacing response signals shown in relation to a temporal framework for pacing response classification in accordance with embodiments described herein;

FIGS. 2A-2B illustrate several types of cardiac pacing response signals shown in relation to a temporal framework including a back-up pace and two blanking periods for pacing response classification in accordance with embodiments described herein;

FIGS. 3A-3E show various arrangements of the temporal framework for pacing response classification;

FIG. 4 is a flow diagram that illustrates a method of operating a cardiac device to adapt classification intervals for cardiac response classification;

FIG. 5 is a flow diagram that illustrates a method of initializing intervals for cardiac response classification that is based on detection of multiple cardiac features;

FIG. 6A-6G illustrate initializing timing parameters of elements of the temporal framework based on a temporal event of a pacing response signal;

FIGS. 7A-7H shows a variety of examples of adapting classification intervals;

FIG. 8 is a flow diagram illustrating a method of initializing a temporal framework for pacing response classification in accordance with embodiments described herein

FIG. 9 presents a flow diagram of a method for determining if the present timing of a signal feature of interest has shifted from its initial timing using a threshold;

FIGS. 10A and 10B are diagrams illustrating a time shifted pacing response feature;

FIG. 11 is a flow diagram illustrating a method for determining if the present timings of a first and second signal feature of interest have shifted from their initial timings;

FIG. 12 is a flow diagram of a method for determining if the present timing of a signal feature of interest has shifted from its initial timings using two thresholds;

FIG. 13 illustrates a temporal framework for left ventricular pacing response classification that includes three classification intervals;

FIG. 14 is a flow diagram illustrating a method of classifying a pacing response of the left ventricle;

FIG. 15 is a flow diagram illustrating classification of a left ventricular pacing response;

FIG. 16 shows a cardiac rhythm management system including an implantable cardiac device (ICD) and lead system that may be used to implement cardiac response classification methods; and

FIG. 17 is a block diagram of the circuitry of the implantable cardiac device according to embodiments described herein.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DESCRIPTION OF VARIOUS EMBODIMENTS

In the following description of the illustrated embodiments, references are made to the accompanying drawings forming a part hereof, and in which are shown, by way of illustration, various embodiments by which the invention may be practiced. It is to be understood that other embodiments may be utilized, and structural and functional changes may be made without departing from the scope of the present invention.

Systems, devices or methods disclosed herein may include one or more of the features, structures, methods, or combinations thereof described herein. For example, a device or system may be implemented to include one or more of the advantageous features and/or processes described below. It is intended that such device or system need not include all of the features described herein, but may be implemented to include selected features that provide for useful structures and/or functionality. Such a device or system may be implemented to provide a variety of therapeutic or diagnostic functions.

After delivery of a pacing pulse to a heart chamber, various cardiac responses to the pacing pulse are possible. In one scenario, the pacing pulse may generate a propagating wavefront of depolarization resulting in a contraction of the heart chamber. In this scenario, the pacing pulse is said to have captured the heart chamber. Capture of the heart chamber may occur if the pacing pulse has sufficient energy and is delivered during a non-refractory period. If the pacing pulse does not produce contraction of the chamber, the cardiac response is referred to as non-captured beat. Non-capture may occur, for example, if the pacing pulse energy is too low, and/or if the pacing pulse is delivered during a refractory period of the cardiac tissue. Fusion occurs when a depolarization initiated by a pace merges with an intrinsic depolarization.

The cardiac pacing response may be determined using a variety of approaches. For example, the cardiac signal sensed following a pacing pulse may be evaluated to discriminate between various pacing responses, e.g., noncapture, capture, fusion, and noncapture with intrinsic activation. Cardiac pacing response classification depends on consistent signal morphology of cardiac signals following the pacing pulse. In some implementations, where feature timing is relatively consistent, the expected timing of the features used for cardiac response determination may be established by the system based on the previous cardiac cycles. For a particular patient, the system may “learn” to expect certain features to occur around a particular time after delivery of the pacing pulse based on the historical timing of the features over a number of previous cardiac cycles.

Discrimination between different types of possible cardiac pacing responses may depend on the timing of expected features that are sensed within a temporal framework. The temporal framework may include classification intervals, blanking periods and appropriately timed back up paces. The classification intervals and blanking periods of the temporal framework are intervals of time that have time parameters that include start time, end time, and length. The relationships and timing parameters of the elements of the temporal framework, e.g., blanking periods, classification intervals, delay periods, and backup pacing, should support detection of features used to discriminate between different types of pacing responses. As the system learns the morphology of the particular patient by analyzing the waveform of the pacing response signal, the temporal framework for pacing response determination may be adjusted to accommodate the individual patient.

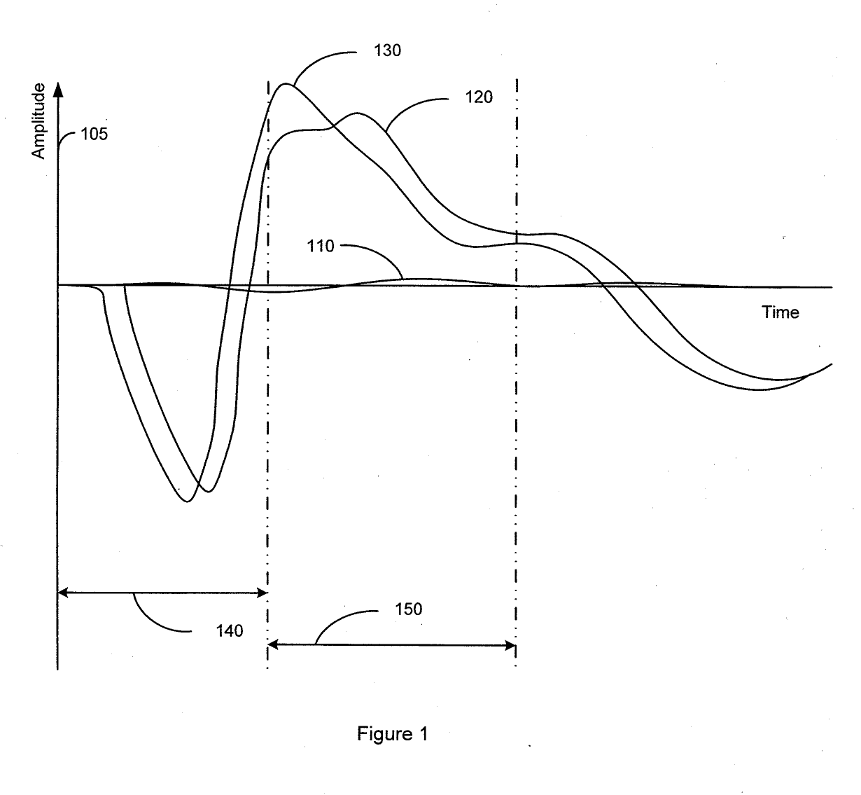

FIG. 1 illustrates several types of cardiac pacing response signals shown in relation to a temporal framework for pacing response classification in accordance with embodiments described herein. FIG. 1 shows pacing response signals indicative of a non-captured response 110, a fusion response 120, and a captured response 130. First and second cardiac classification intervals 140, 150 superimposed over the response signals 110, 120, 130 are also shown in FIG. 1. The first cardiac classification interval 140 begins after a pacing pulse 105 to a heart chamber. The pacing pulse 105 may be delivered to any heart chamber and the pacing response signal of the paced heart chamber is sensed. For example, the pacing pulse 105 may be delivered to any of the left ventricle, the right ventricle, the left atrium, or the right atrium. The second classification interval 150 may begin after the first classification interval 140 or a delay period may be interposed between the first classification interval 140 and the second classification interval 150. In some cases, the first classification interval 140 and the second classification interval 150 may overlap in time.

The classification intervals 140, 150 define time periods in which features of the pacing response signal can be detected to classify the heart chamber's response following the pacing pulse 105. For example, the pacing response signal may indicate that the heart chamber's response following the pacing pulse 105 is non-capture, fusion, capture, or intrinsic. The features which indicate the pacing response may be one or a combination of positive peaks, negative peaks, slopes, inflection points, zero crossing, and/or other cardiac signal features. In some embodiments, if the cardiac signal sensed in the first classification interval 140 preliminarily indicates capture, the second classification interval 150 may be used to look for at least one feature to verify that capture has occurred. In some cases, more than two classification intervals may be used to classify the pacing response signal.

Each classification interval 140, 150 may be bounded in time and can be characterized by timing parameters including the start time of the classification interval, the length of the interval, and/or the end time of the interval. The timing parameters of the first interval 140 and the second interval 150, and/or the timing relationship between the first and second intervals 140, 150 may be established for cardiac response classification based on the timing of one or more pacing response signal features within at least one of the classification intervals 140, 150. The timing relationship between the elements of the temporal framework may or may not involve a cause and effect relationship. For example, each of classification intervals, delay periods, blanking periods, and back up pacing may be considered temporal framework elements. If the timing of one of these elements is modified, this timing change may necessitate a timing change in the timing of one or more other element.

In some cases, delaying the start time of the first interval 140 may also have the effect of delaying the start time of the second interval 150. Alternatively, the length of the first interval 140 could be shortened as well as the start time delayed and the second interval 150 may remain unchanged. The two intervals 140, 150 could also overlap in time allowing the second interval 150 to start at its original starting time even if the first interval 140 is delayed or lengthened. Timing parameters of the second interval 150 may also be changed and may have an effect on the timing parameters of the first interval 140. According to some embodiments, the timing parameters of the first interval 140 and the timing parameters of the second interval 150 are determined independently of one another.

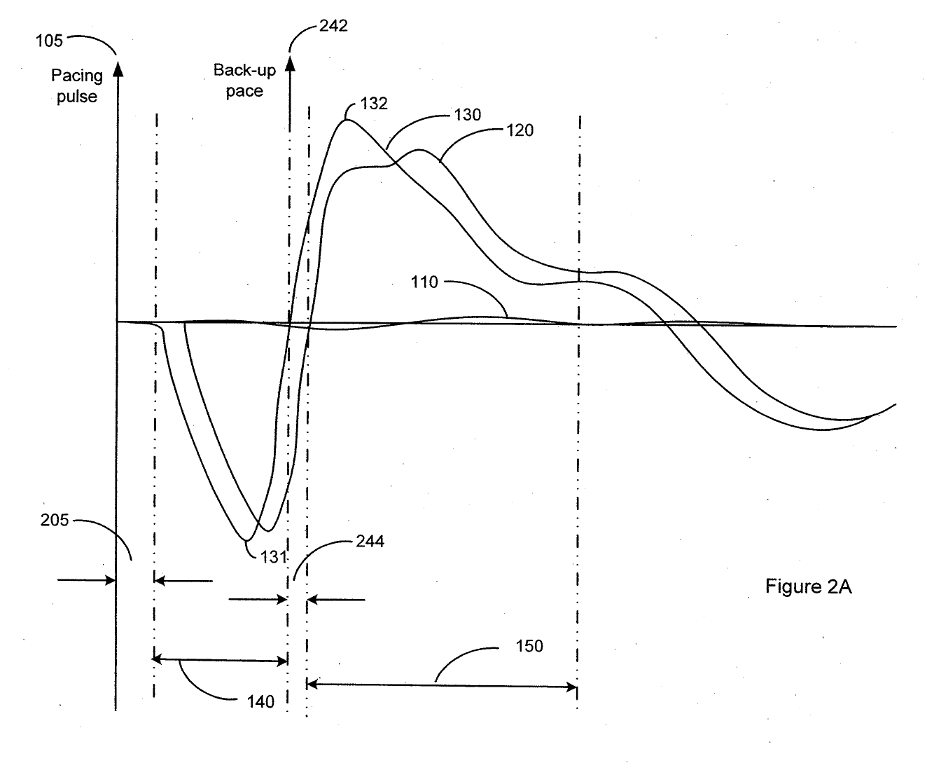

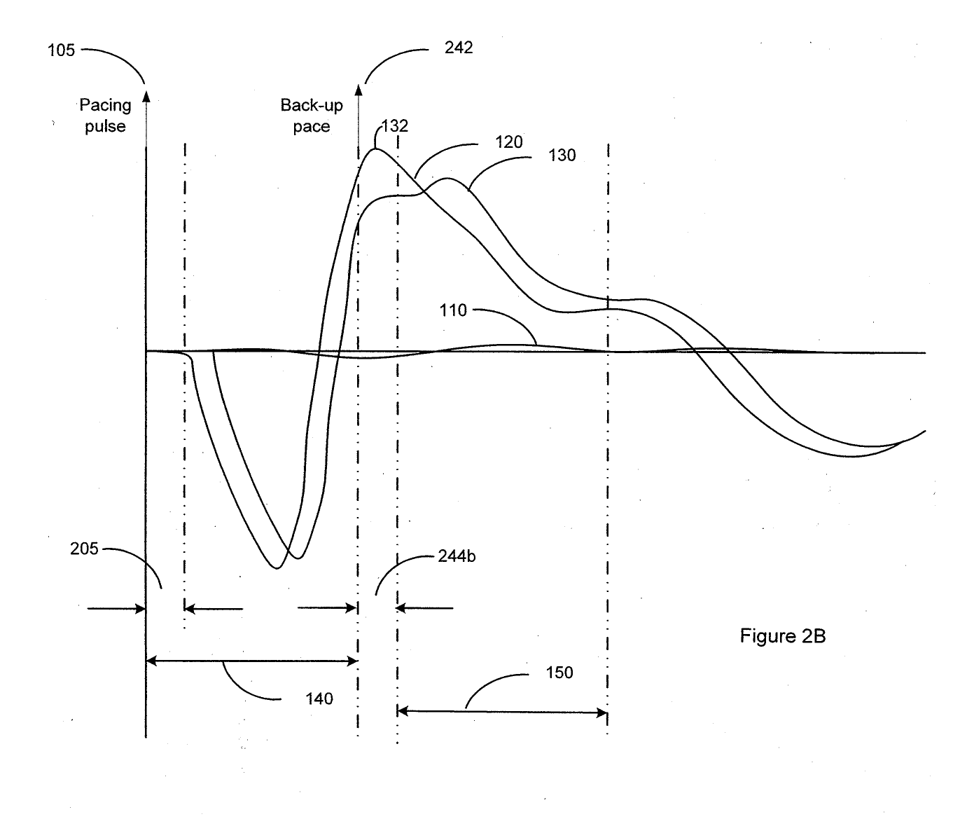

FIG. 2A is similar to FIG. 1 in some respects with the addition of two blanking periods 205, 244 and the addition of a back-up pacing pulse 242. FIG. 2A illustrates the same cardiac pacing response signals 110, 120, 130 shown in FIG. 1 that are indicative of a non-captured response 110, a fusion response 120, and a captured response 130. These signals are displayed in relation to the temporal framework that includes the cardiac response classification intervals 140, 150, blanking periods 205, 244, and back up pace 242 in accordance with embodiments described herein.

In the embodiment illustrated in FIG. 2A, the sense channel used to sense the pacing response signal is blanked during a first blanking period 205 that follows the pacing pulse 105. Blanking is implemented on the sense channels to prevent the very large electrical signal generated by the pacing pulse from reaching the input circuitry of the sense channels. During the blanking periods 205, 244, the sense channel may be disconnected or the sensing ability of the sense channel may be otherwise disabled. Various timing parameters of the first blanking period 205 may be adjusted. For example, the length and/or the start time of the first blanking period 205 may be adjusted to allow sensing of cardiac signal features of interest.

The first blanking period 205 may be implemented on sense channels that sense in the same chamber as the pacing pulse 105. Blanking periods may additionally or alternatively be implemented on sense channels that sense in one or more other heart chambers, e.g., the contralateral heart chamber and/or the ipsilateral heart chamber to the chamber being paced. As shown in FIG. 2A, the first blanking period 205 may begin immediately after the pacing pulse 105. In some embodiments, the first blanking period 205 may begin after a short delay period following the pacing pulse 105. Alternatively, the first blanking period 205 may begin before the pacing pulse 105.

Because sensing is disabled during blanking, a blanking period that is too long may prevent features of the pacing response signal from being detected. In these cases, the length of the blanking period may need to be modified to avoid undersensing of signal features.

In FIG. 2A, the first interval 140 begins immediately after the first blanking period 205 and extends after the first blanking period 205. According to some implementations, a delay period could be interposed between the first blanking period 205 and the first classification interval 140. Alternatively, in some cases, the first classification interval 140 may temporally overlap with the first blanking period 205. Even though there is no sensing during a blanking period, a blanking period may still occur within a classification interval. Sensing would not occur during the portion of the classification interval containing the blanking period.

The approaches described herein are particularly useful for capture threshold testing and may also be used during therapeutic pacing (non capture threshold testing) with capture verification. During a capture threshold test and/or automatic capture verification, back up paces may be delivered to ensure continued pacing support in the event of persistent non-capture. In some cases, back-up pacing is applied only if noncapture is detected after an initial pace. In some capture threshold test implementations, a back-up pace is delivered after every test pace of the capture threshold test.

The implementation shown in FIG. 2A includes a back-up pacing pulse 242 that is delivered to the heart chamber after the end boundary of the first classification interval 140. The back-up pacing pulse 242 may be delivered to the heart chamber paced by the pacing pulse 105 or may be delivered to the contralateral heart chamber, for example. The back-up pace 242 is delivered at an energy previously determined to be sufficient for capture. For example, the back-up pace may be delivered using a preset energy or an energy that has been adjusted based on one or more previous capture threshold tests. Back-up pacing based on a previous capture threshold test may be useful for energy conservation, for example. The back-up pace 242 may be delivered immediately after the first classification interval 140 ends or may be delivered following a delay period after the first classification interval 140 ends. The time of back-up pace 242 may define the end boundary of the first classification interval 140 and/or may define the start boundary of the second blanking period 244, for example.

In some approaches, the timing of the back-up pace 242 and the timing parameters of the second blanking period 244 may be determined based on the timing of one or more cardiac signal features indicative of the cardiac pacing response. For example, the back-up pace 242 may be delivered before cardiac signal features indicative of capture, fusion or a non-captured/intrinsic response are expected to occur. The length and/or the start time of the second blanking period 244 that follows the back-up pace 242 may be adjusted to facilitate sensing of the cardiac features used for pacing response classification.

A second classification interval 150 follows the second blanking period 244 in FIG. 2A. The second classification interval 150 may begin immediately after the second blanking period 244 ends or the second classification interval 150 may begin after a delay period following the second blanking period 244. In some cases, the second classification interval 150 may also begin before the second blanking period 244 ends.

As described in connection with FIG. 1, the classification intervals 140, 150 define time intervals in which features of the pacing response signal can be detected to classify a cardiac pacing response signal as non-capture, fusion, or capture. The features used for pacing response classification may be positive or negative peaks, slopes, inflection points, and/or other signal features. In some embodiments, if the cardiac signal sensed in the first classification interval 140 preliminarily indicates capture, the second classification interval 150 may be used to look for at least one feature to verify that capture has occurred. In some cases, more than two classification intervals may be used to classify the pacing response signal.

Each classification interval 140, 150 and blanking period 205, 244 can be characterized by timing parameters including the start time of the interval or period, the length of the interval or period, and/or the end time of the interval or period. The back-up pace 242 is also characterized by the time of the back-up pace event 242. The temporal framework, including the timing parameters of the classification intervals 140, 150, blanking periods 205, 244, and back-up pace 242, as it is imposed on a pacing response signal, can determine the effectiveness of the pacing response classification. For example, if a blanking period is too long, features of interest may not be detected. In another example, a classification interval may be too short, missing important response signal features.

The classification intervals 140, 150, the blanking periods 205, 244, and the back-up pace 242 are events that can be temporally related. The temporal relationships between these events affect successful pacing response classification. Changing a timing parameter of one event may cause a change in the timing of other events. For example, delaying the start time of the first classification interval 140 may also have the effect of delaying the time of the back-up pace 242. Delaying the back-up pace may also delay the start time of the second blanking period 244, and/or the second classification interval 150. As another example, if the first classification interval is shortened, the timing parameters of the first blanking period 205 may remain unchanged or the first blanking period 205 may also be shortened. Shortening the first classification interval 140 may move the start time of the back-up pacing pulse 242. According to some embodiments, the timing parameters of two or more of the first classification interval 140, the second classification interval 150, the first blanking period 205, the second blanking period 244, and the back-up pace 242 are not temporally related and may be determined independently of one another.

FIG. 2A illustrates an example in which the first peak 131 of the captured response signal 130 falls within the first classification interval 140 and the second peak 132 falls within the second classification interval 150 allowing the peaks 131, 132 to be detected and resulting in appropriate pacing response classification. FIG. 2B is similar to FIG. 2A with an adjustment of the timing parameters of some elements of the temporal framework for pacing response classification. For example, FIG. 2B illustrates an instance where the timing of the second blanking period 244b prevents detection of the second peak 132. This has the effect of suppressing sensing of the second peak and could result in a misclassified pacing response. In this example, it may be beneficial for pacing response classification to shorten the first classification interval and to move the back-up pace 242 and second blanking period 244b to an earlier time as in FIG. 2A that will allow the second peak to be sensed.

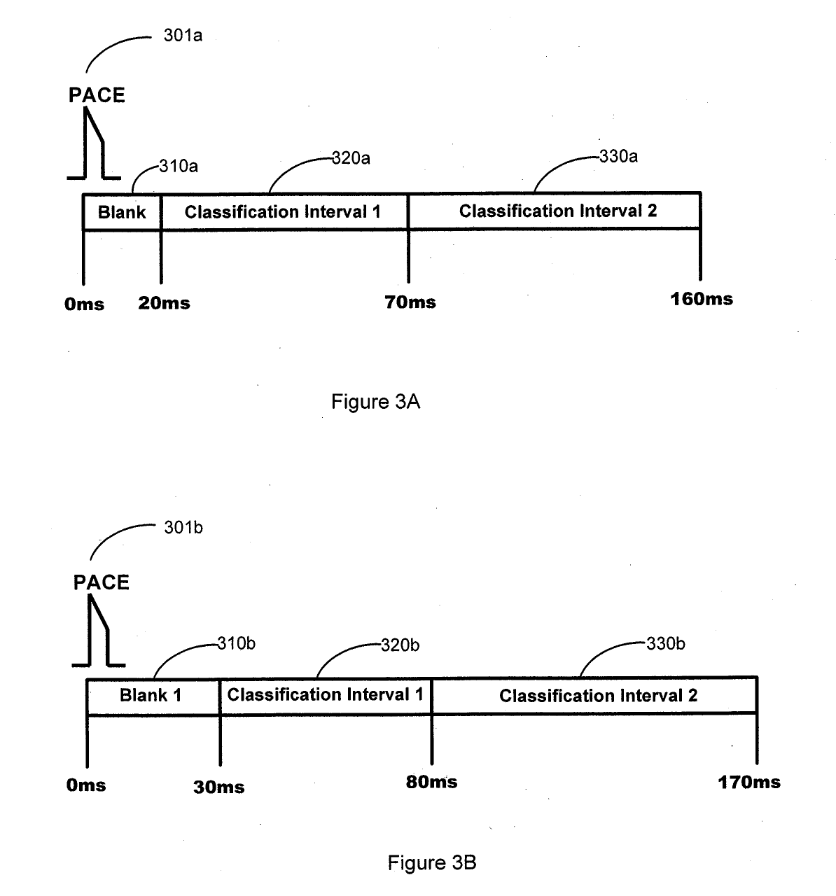

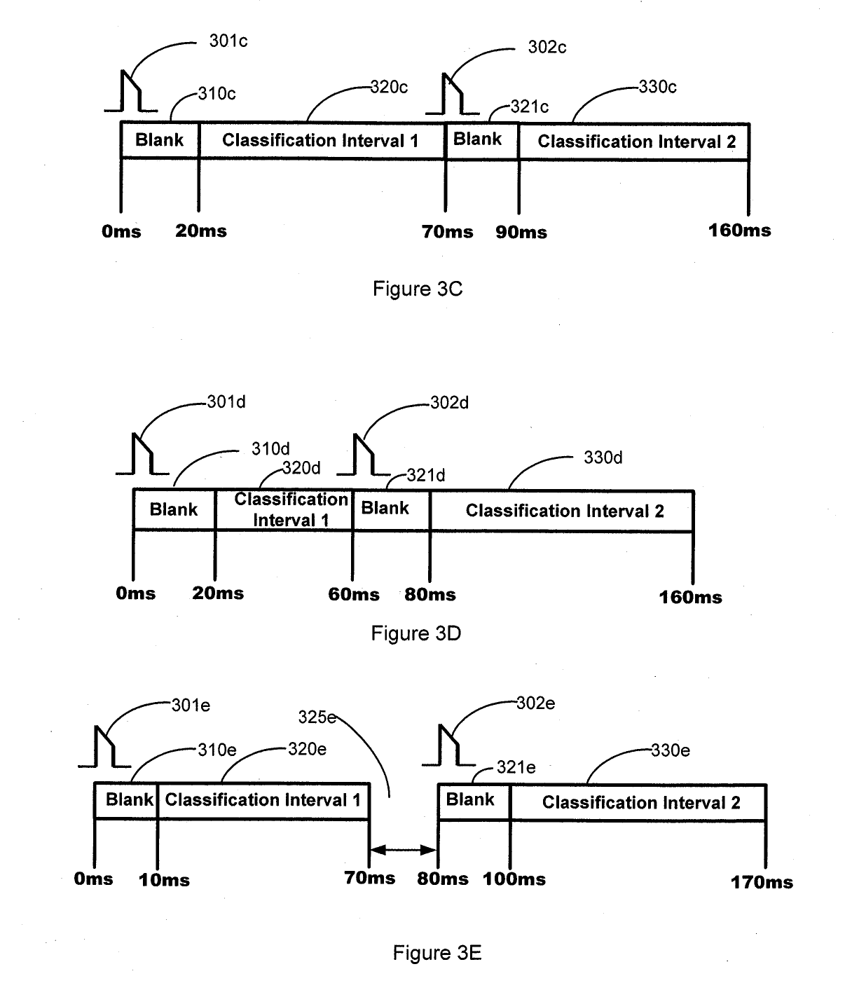

FIGS. 3A-3D show various arrangements of the temporal framework for pacing response classification. The temporal frameworks illustrated in FIGS. 3A-3D show different scenarios of timing parameters for the boundaries of the pacing response classification intervals, blanking periods and delay periods and the relationships between these temporal framework elements. FIG. 3A shows a pacing pulse 301a followed by a first blanking period 310a, a first classification interval 320a, and a second classification interval 330a. In this example, each of the intervals 310a, 320a, 330a extend continuously in that there are no gaps between any two of the intervals/periods 310a, 320a, 330a. The intervals/periods 310a, 320a, 330a also do not overlap in time. In various implementations, blanking period 310a may have a range from about 0 milliseconds (ms) to about 100 ms, or a range from about 0 ms to about 37.5 ms. For example, the blanking period 310a illustrated in FIG. 3A is about 20 ms. A delay period (not shown) may follow the blanking period 310a and the delay period may have a range from about 0 ms to about 200 ms, or a range from about 0 ms to about 157.5 ms. The first classification interval 320a may have a range of about 0 ms to about 400 ms, or a range of about 0 ms to about 317.5 ms. For example, the first classification interval 320a illustrated in FIG. 3A is about 50 ms. The second classification interval 330a may start immediately after the first classification interval 320a ends and may have a range of about 0 ms to about 400 ms, or a range of about 0 ms to about 317.5 ms. For example, as illustrated in FIG. 3A, the second classification interval 330a may be about 90 ms. In some instances there may be a delay period (not shown) between the blanking period 310a and the first classification interval 320a and/or the first classification interval 310a and the second classification interval 330a. Additionally, in some embodiments, there may be a back-up pace and a second blanking period after the first classification interval 320a and before the second classification interval 330a.

FIG. 3B shows another example of a temporal framework in accordance with embodiments described herein. As in the example illustrated in FIG. 3A, the blanking period 310b begins at the time of the pacing pulse 301b and extends about 10 ms longer than the blanking period 310a of FIG. 3A. Unlike the example of FIG. 3A, the first classification interval 320b is delayed by 10 milliseconds in FIG. 3B due to the delay in the blanking period 310b. The second classification interval 330b is also delayed by 10 ms as a result of the 10 ms delay of the blanking period 310b. This scenario could occur, for example, if an unexpected peak that is not of interest in pacing response classification occurs after the blanking period 310b within the first classification interval 320b. For example, such an unexpected peak may arise when certain leads or sensing vectors are used. An unexpected peak may cause erroneous pacing response classification to occur. The blanking period may be adjusted to prevent sensing of the unexpected peak. In some cases an unexpected peak may be avoided by adding a delay period between a first classification interval and a first blanking period. Note that one or more delay periods may be interposed between any of intervals of the temporal framework.

FIG. 3C illustrates yet another temporal framework representing another example of intervals and periods used in pacing response classification. This temporal framework is similar in some respects to the temporal framework of FIG. 3A and includes a pacing pulse 301c, a blanking period 310c and first and second classification intervals 320c, 330c. The temporal framework of FIG. 3C differs from that of FIG. 3A because a back up pace 302c and a corresponding blanking period 321c are also included in the temporal framework. The second blanking period 321c may have a range from about 0 ms to about 37.5 ms, for example. In the case of FIG. 3C the second blanking period has a duration of 20 ms.

In FIG. 3D the first classification interval 320d is shortened relative to the first classification interval 320c of FIG. 3C by maintaining the start time of the first classification interval 320d and moving the end time of the first classification interval 320d to an earlier time. The timing parameters for the first blanking period 310d remain the same as in FIG. 3C. In this case, the backup pacing pulse is moved to an earlier time and the start time of the second blanking period 321d also moves to an earlier time. In this example, there is no delay between the end time of the first classification interval 320d and the start time of the second classification interval 330d although that scenario is also possible.

FIG. 3E shows another temporal framework that may be implemented if the first blanking period is too long, causing the first classification interval to start too late to detect the feature of interest. To correct this deficiency, the blanking period 310e that follows the pacing pulse 301e may be shortened and the start time of first classification interval 320e is moved up to an earlier time. In this case, the timing parameters of the backup pace 302e, the second blanking period 321e, and the second classification interval 330e remain the same so that the start time, end time, and the length of the second classification interval 330e remain the same as in FIG. 3C. This causes a temporal gap (delay period) between the first classification interval 320e and backup pace 302e and start of the second blanking period 321e. In other examples, the backup pace and start of the second blanking period may be moved up to an earlier time so that there is no delay between the end time of the first classification interval and the start time of these events.

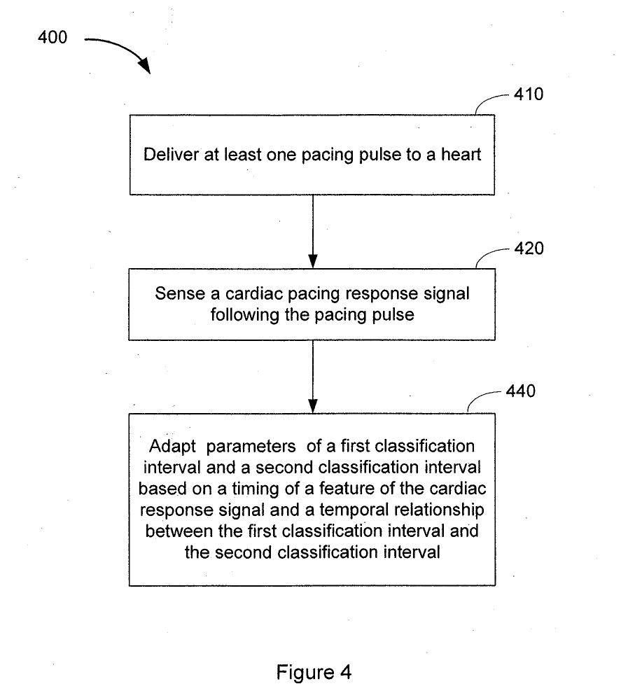

Adaptation of the temporal framework elements may enhance pacing response classification. FIG. 4 is a flow diagram that illustrates a method 400 of operating a cardiac device to adapt classification intervals for cardiac response classification in accordance with various embodiments described herein. At least one pacing pulse is delivered to a heart 410. A cardiac pacing response signal is sensed 420 following the pacing pulse in one or both of the first classification interval and the second classification interval. Parameters of the first classification interval and the second classification interval are adapted 440 based on a timing of a feature of the cardiac response signal and a temporal relationship between the first classification interval and the second classification interval.

The elements of the temporal framework used in pacing response classification may be adjusted so that features of interest fall appropriately within the classification intervals and are not blanked by the blanking periods. For example, adjustment of the intervals and/or blanking periods and/or other elements can be based on the feature timing for a particular type of pacing response to achieve optimal feature detection. In some cases, pacing response classification is achieved based on one or multiple features of the pacing response signal. The use of cardiac response features for response classification is further described in commonly owned U.S. Pat. Nos. 7,319,900, 7,774,064, 7,337,000, 7,499,751, and 7,574,260, which are incorporated herein in their respective entireties.

Initialization of the classification intervals may occur prior to or during use of the temporal framework to determine the cardiac pacing response. For example, the initialization may be performed fully automatically by the cardiac pacing device, or may be performed partially automatically by a physician operating a device programmer to program the pacing device with appropriately timed intervals/periods. For example, a pacing device and/or device programmer may suggest or indicate appropriate timing parameters for the classification intervals/delay periods that can be accepted by a physician to be programmed into the cardiac pacing device.

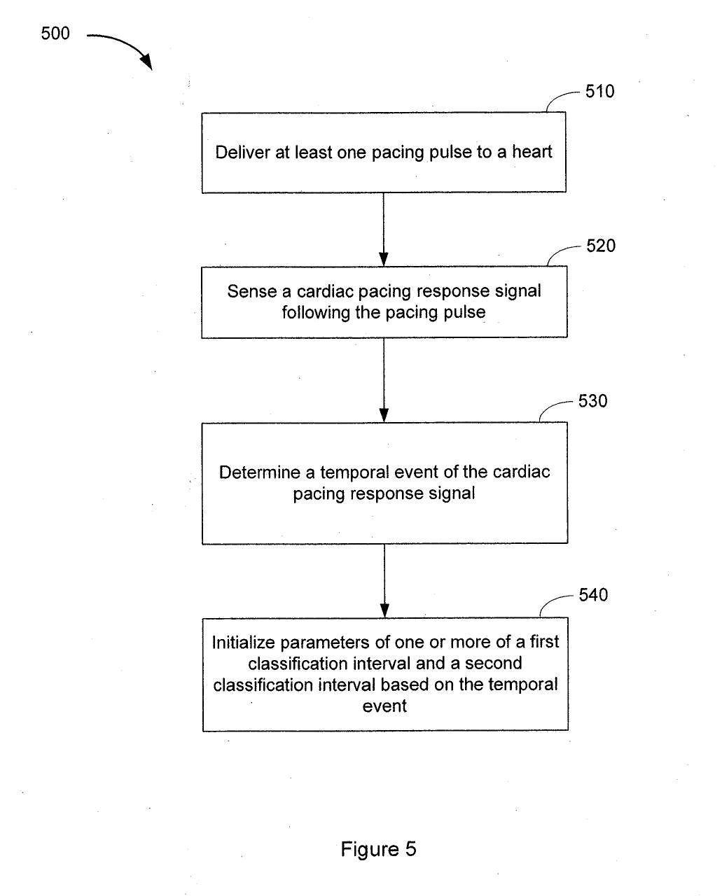

FIG. 5 is a flow diagram that illustrates a method 500 of initializing intervals for cardiac response classification that is based on detection of multiple cardiac features in accordance with various embodiments described herein. In these examples, the initialization may include determining a pacing response signal, e.g., an electrogram (EGM), including multiple samples of the response signal during a cardiac cycle. In some applications, the EGM may store cardiac signal samples at a rate of about 400 samples/sec. These samples are used in the initialization of the pacing response temporal framework such as the classification intervals and/or blanking periods. The method 500 includes delivering 510 at least one pacing pulse a heart chamber. The method 500 of FIG. 5 further includes sensing 520 a cardiac pacing response signal of the heart chamber to which the pacing pulse is delivered following delivery of the pacing pulse. During the initialization, at least one temporal event of the cardiac signal is detected. For example, the temporal event may be a zero crossing point, an inflection point, the time of occurrence of a peak (or other feature) of the response signal, a temporal distance between two features of the response signal, or other temporal event. In some cases the zero crossing point and/or inflection point may be obtained from a multi-sample EGM of a captured response signal.

Timing parameters of one or more of a first classification interval and a second interval are then initialized 540 based on the temporal event. For example, in some cases, the timing parameters of the classification intervals/delay periods may be initialized so that a first feature of the pacing response signal falls within the first classification interval and the second feature falls within the second classification interval. In some cases, an end and/or start time of a classification interval and/or blanking period may be initialized based on a zero crossing point, an inflection point, or some other temporal event of the pacing response signal. In yet other cases, the timing parameters of classification interval and/or blanking period are calculated based on a temporal distance between a feature of the cardiac response signal and the start and/or end time of the classification interval and/or blanking period. After initialization, the parameters are stored for future use in capture verification during therapeutic pacing and/or for use in capture threshold testing.

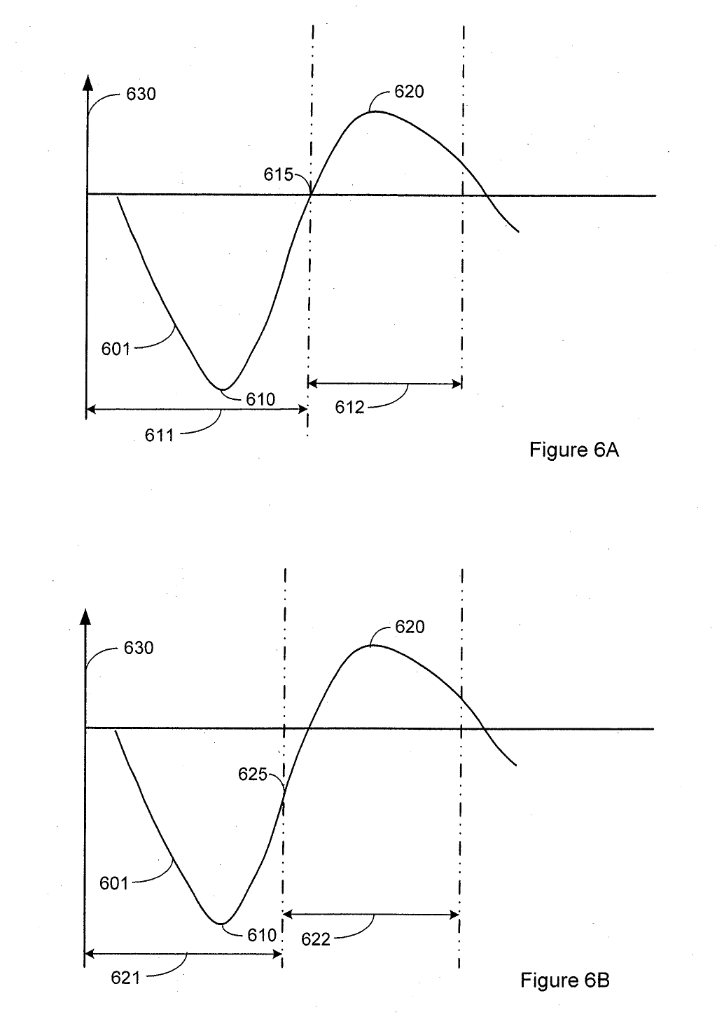

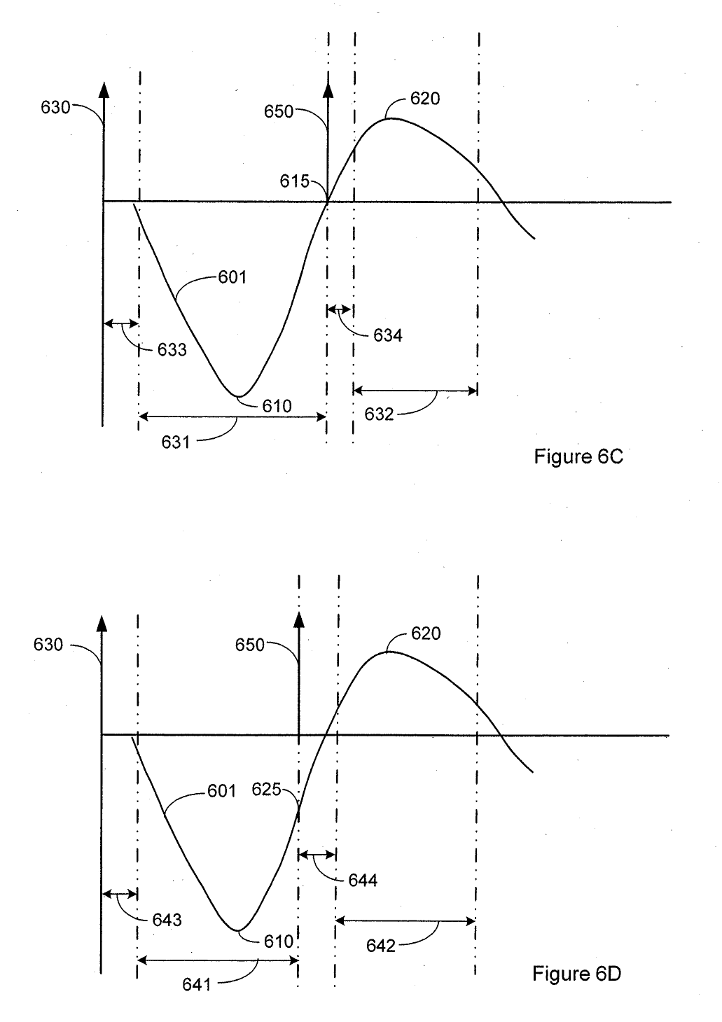

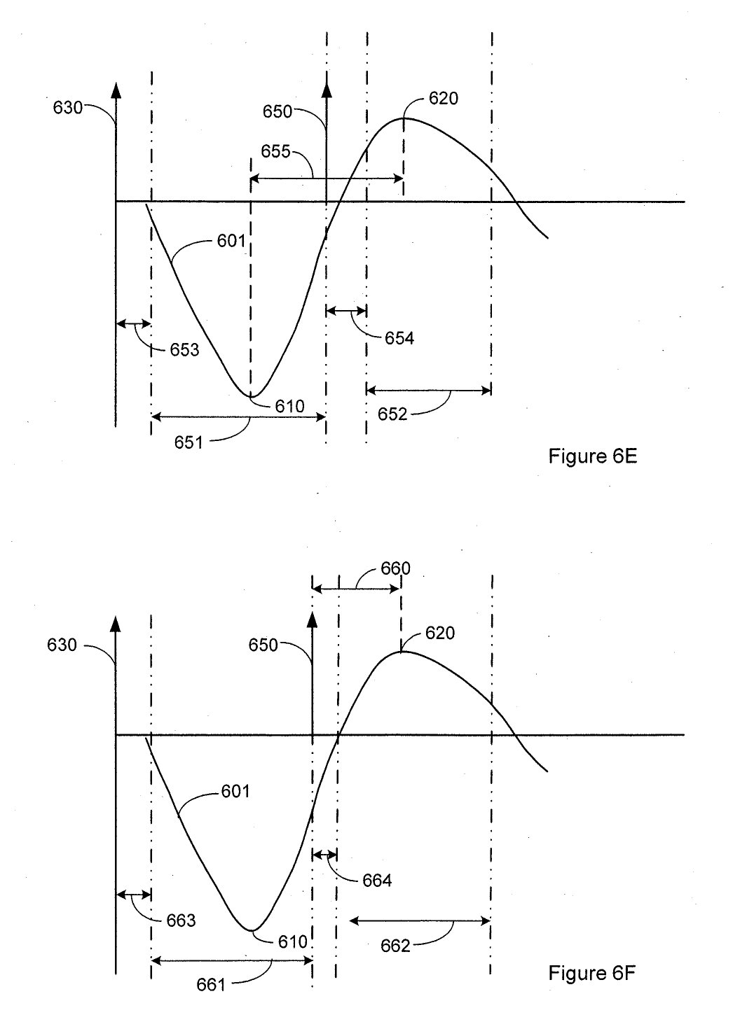

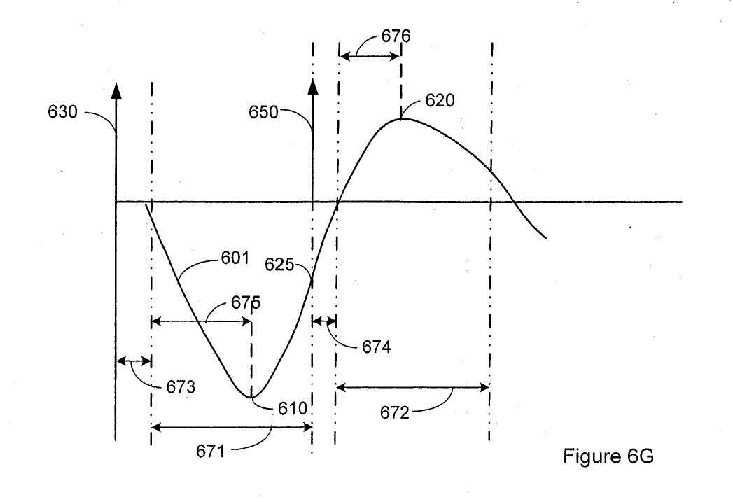

FIGS. 6A and 6B illustrate initializing first and second classification intervals based on a temporal event of a pacing response signal 601 that follows a pacing pulse 630. In the examples illustrated in FIGS. 6A and 6B, the end time of the first classification interval 611, 621 and the start time of the second classification interval 612, 622 are established to coincide with the time of occurrence of the temporal event 615, 625. In these examples, the end time of the first classification interval and the start time of the second classification interval are set to correspond to the time of the temporal even 615, 625. In FIG. 6A, the temporal event 615 is the zero crossing point of the pacing response signal. In FIG. 6B, the temporal event 625 is an inflection point and/or midpoint between first and second peaks of the pacing response signal. In each example, setting the end time of the first classification interval 611, 621 and the start time of the second classification interval 612, 622 based on the timing of the temporal event 615, 625 causes a first feature 610 of the pacing response signal 601, e.g., first peak, to fall within the first classification interval 611, 621 and a second feature 620 of the pacing response signal 601, e.g., second peak, to fall within the second classification interval 612, 622.

In some cases, the timing of the temporal event may be used to establish the timing of a back up pace and a corresponding blanking period, as illustrated in FIGS. 6C and 6D. FIGS. 6C and 6D include a first blanking period 633, 643 that occurs after the primary pace 630 and a blanking period 634, 644 that occurs after the back up pace 650. In FIGS. 6C and 6D, the timing of the back up pace 650, the end time of the first classification interval 631, 641, and start time of the following blanking period 634, 644 are determined by the temporal event 615, 625. In FIG. 6C, the temporal event 615 is the zero crossing point and in FIG. 6D, the temporal event 625 is an inflection point of the cardiac signal.

In some implementations, the temporal event used to determine the timing of the back up pace, classification intervals and/or blanking periods is a point in time between the time coordinates of two cardiac signal features, which may or may not be the same features used to classify the pacing response. In FIG. 6E, the time of the back up pace 650, the end time of the first classification interval 651 and the start time of the second blanking period 654 are set to correspond to the midpoint in time and/or amplitude between negative and positive peaks 610, 620 of the pacing response signal 601. In some implementations, the time of the back up pace, the end time of the first classification interval and the start time of the second blanking period are set to occur at predetermined offset from a feature of the cardiac signal. For example, in FIG. 6F, the time of the back up pace 650, the end time of the first classification interval 661 and the start time of the second blanking period 664 are set to occur at a predetermined interval 660 from a positive peak 620 of the cardiac signal 601. Alternatively, the timing of the backup pace 650 and/or timing parameters of the classification intervals and/or blanking periods may be established based on the negative peak 610 of the cardiac signal 601 and/or other features of the cardiac signal 601.

In some cases, multiple temporal events may be used in establishing the backup pace timing and/or parameters of the classification intervals and/or blanking periods. For example, in the implementation illustrated in FIG. 6G, the back up pace timing, the start time of the second blanking period 674 and the end time of the first classification interval 671 are determined based on the inflection point 625 of the cardiac signal 601. The end time of the first blanking period 673 and the start time of the first classification interval 671 are set to occur at a predetermined interval 675 before the time of occurrence of a first signal feature, in this case, the negative signal peak 610. The end time of the second blanking period 674 and the start time of the second classification interval 672 are set to occur at a predetermined interval 676 before the time of occurrence of a second signal feature, in this case, the positive signal peak 620. Thus, in this example, the multiple temporal events used to initialize the temporal framework include the inflection point 625 of the cardiac signal 601, the first peak 610 of the cardiac signal 601, and the second peak 620 of the cardiac signal 601.

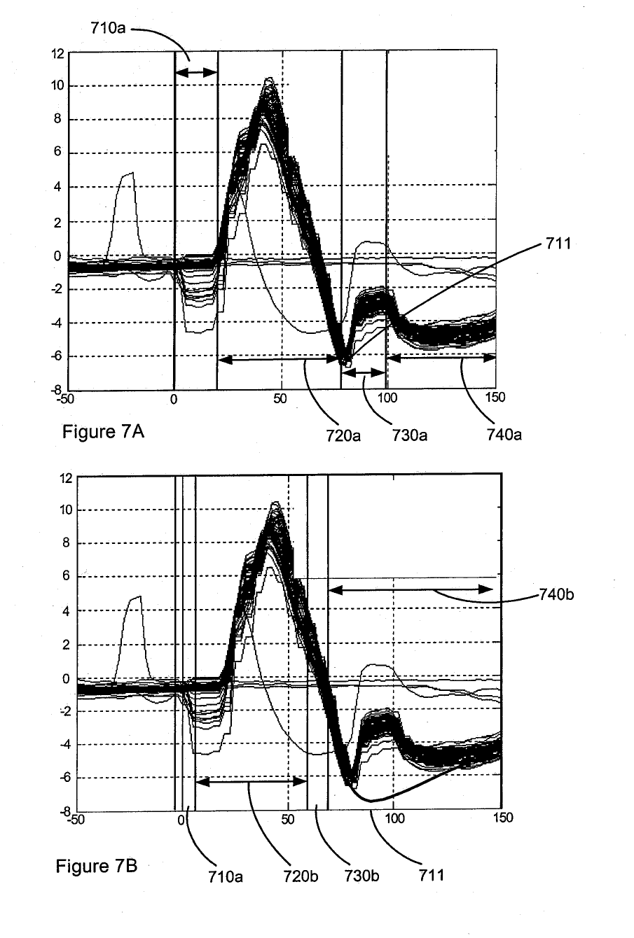

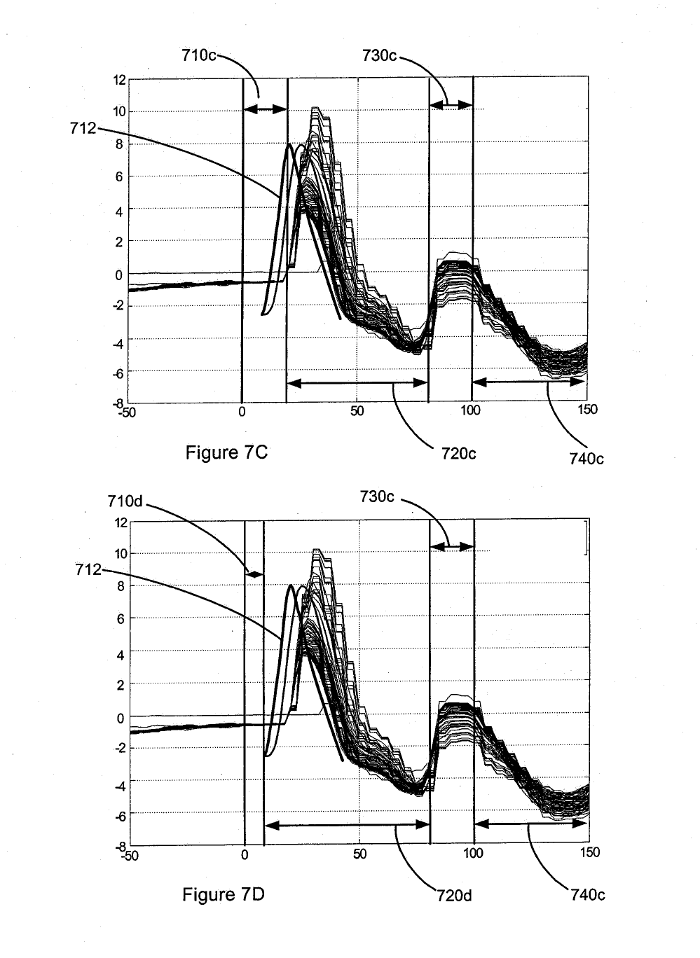

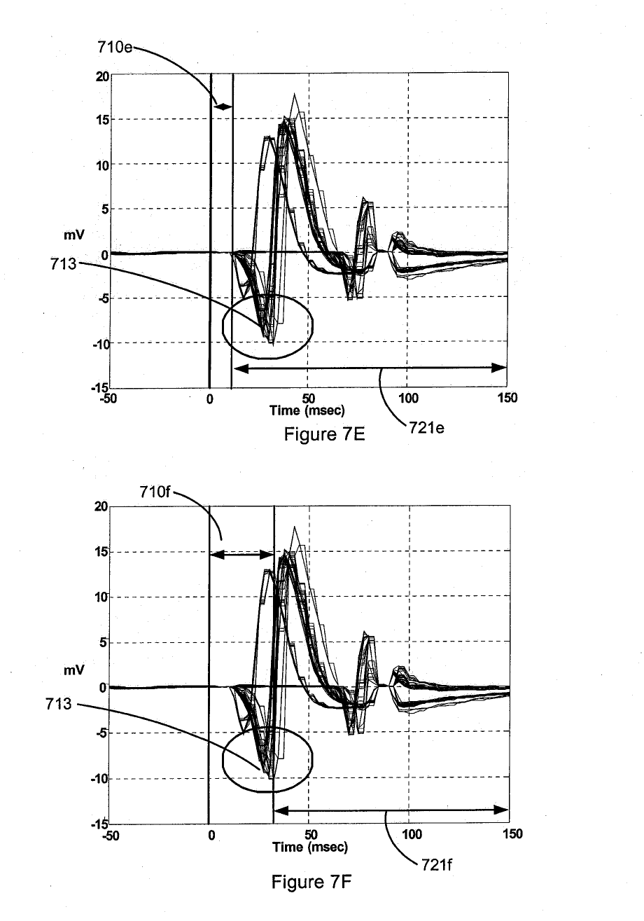

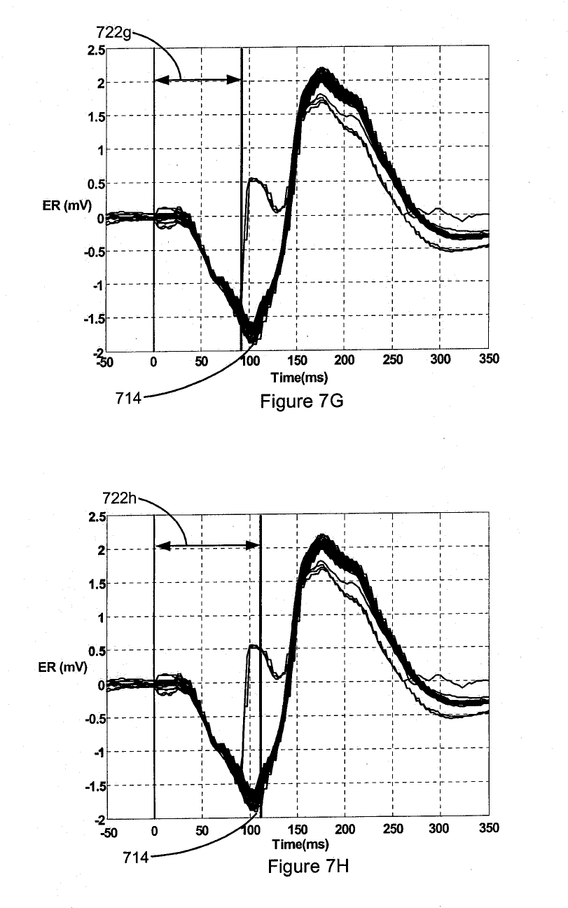

Initialization and the adjustment of timing parameters of elements of the temporal framework may help to achieve more accurate pacing response classification. FIGS. 7A-7H display examples of situations of inaccurate pacing response classification due to inadequate timing parameters of elements of the temporal framework. For example, FIG. 7A displays a pacing response signal with a temporal framework superimposed over the response signal. The temporal framework, in this example, includes a first blanking period 710a, a first classification interval 720a, a second blanking period 730a, and a second classification interval 740a. As can be observed from FIG. 7A, the second peak 711 occurs within the second blanking period 730a, which prevents the second peak 711 from being sensed and potentially causing inaccurate classification of the pacing response signal. FIG. 7B shows an example of how boundaries of the temporal framework may be adjusted to allow the second peak 711 to be sensed. In FIG. 7B the first classification interval 720b is shortened relative to the first classification interval 720a of FIG. 7A. Shortening the first classification interval 720b has the effect of moving the start time of the second blanking period 730b to an earlier time in relation to the second blanking period 730a of FIG. 7A. The start time of the second classification interval 740b is also moved up in time, allowing the second peak 711 of the pacing response signal to be sensed in the second classification interval 740b.

FIGS. 7C and 7D illustrate another example in accordance with embodiments described herein. FIG. 7C includes a first and a second classification interval 720c, 740c, and a first and a second blanking period 710c, 730c. In this example, the first classification interval 720c starts too late and part of the first peak 712 is within the first blanking period 710c and thus is not sensed. FIG. 7D shows the same pacing response as in FIG. 7C with some changes to the timing parameters of elements of the temporal framework. In this case, the start time of the first classification interval 720d is moved to an earlier time with respect to the first classification interval 720c of FIG. 7C. The change in the start time of the first classification interval 720d causes the duration of the first blanking period 710d to be shortened with respect to the duration of the first blanking period 710c of FIG. 7C. The second blanking period 730c and the second classification interval 740c retain the same timing parameters in FIGS. 7C and 7D. In some cases, the end time of the first classification interval 720d may be moved up in time so that the start times of a second blanking period and a second classification interval are also moved up in time. Moving up the end time of the second classification interval and the start times of the second blanking period and second classification interval facilitates detection of the negative peak of the cardiac signal.

FIGS. 7E and 7F illustrate yet another example of how the timing parameters of elements of the temporal framework can be adjusted so as to allow for more accurate pacing response classification. FIG. 7E includes a blanking period 710e and a classification interval 721e. In this example, an unexpected peak 713 is sensed in the classification interval 721e. Such an unexpected peak may arise when certain leads, lead placements, or sensing vectors are used. An unexpected peak may cause erroneous pacing response classification to occur. FIG. 7F provides an adjustment that prevents sensing of the unexpected peak 713. The start time of the classification interval 721f is delayed relative to the classification interval 721e of FIG. 7E. The delay of the classification interval 721f causes the blanking period 710f to be extended when compared to the blanking period 710e of FIG. 7E and the unexpected peak 713 is no longer sensed. One or more delay periods can be interposed between any elements of the temporal framework to facilitate feature detection.

FIGS. 7G and 7H show another example of how the timing parameters of elements of the temporal framework can be adjusted so as to allow for more accurate pacing response classification. FIG. 7G illustrates a situation where capture is being erroneously classified as non-capture because the peak 714 of the captured response signal is not within the first classification interval 722g. In this instance the response signal does not reach a capture detection threshold and the response is classified as non-capture and a back-up pace may be delivered at, after, or near the end of classification interval 722g. FIG. 7H shows a first classification interval 722h with a longer duration. The end time of the first classification interval 722h is delayed with respect to the end time of the first classification interval 722g of FIG. 7G. The peak 714 is detected in the first classification interval 722h in FIG. 7H because of the delayed end time of the first classification interval 722h and capture is detected.

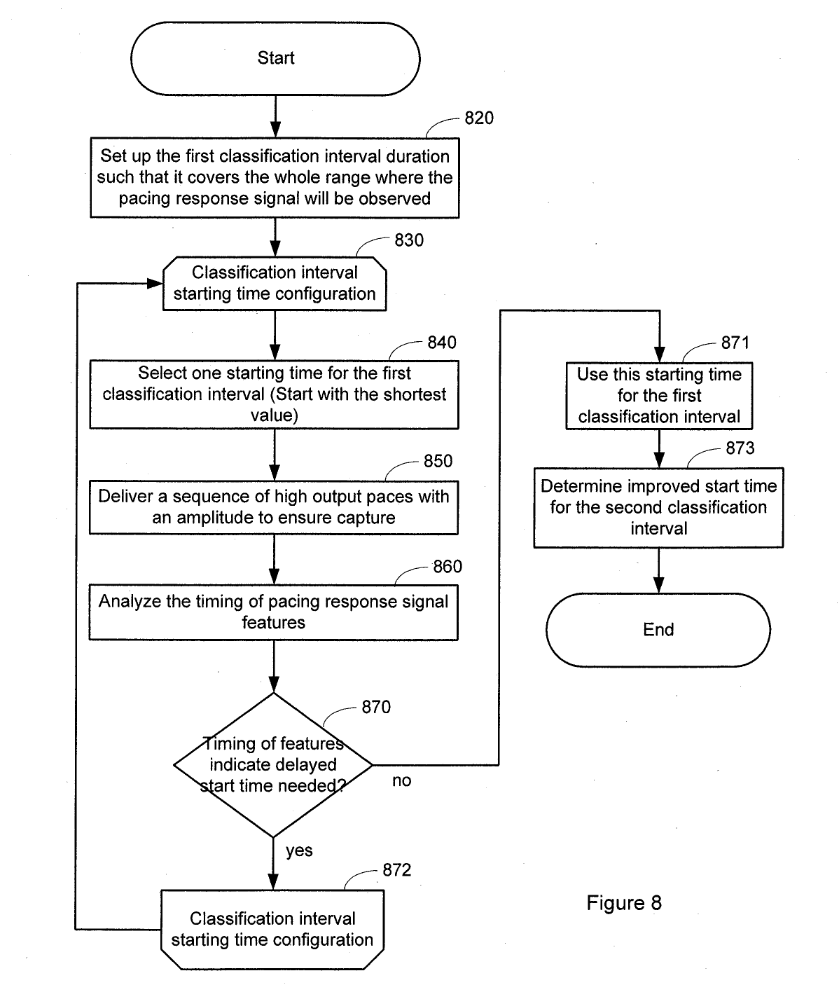

FIG. 8 is a flow diagram illustrating a method for initialization in accordance with some embodiments described herein. The first classification interval duration is set 820 such that the first classification interval covers the whole range where the pacing response signal will be sensed. Configuration of the start time of the classification interval occurs in process loop 830-870. A start time for the first classification interval is selected 840. The first time through process loop 830-870, the shortest value for the classification interval start time is chosen. A sequence of high output paces with an amplitude to ensure capture is delivered 850. The timing of pacing response signal features are analyzed 860. The process determines if the timing of features of the pacing response signal indicate 870 that a delayed start time for the first classification interval is needed. In some implementations, a multi-sample EGM is acquired and analysis of the timing of pacing response signal features is performed on the multi-sample EGM. If the process determines that the timing of features indicate that a more delayed start time is needed, the process loops back to the classification interval start time configuration and continues until timing of features indicate 870 that a more delayed start time for the first classification interval is not needed. If the process determines 870 that a delayed start time is not needed, the current start time for the first classification interval is selected 871. An improved start time for the second classification interval is also determined 873. For example, the start time for the second classification interval may be determined using process 830-870. The improved start time for the second classification interval may or may not depend on the determined start time of the first classification interval. Additional classification intervals may be initialized using this process.

After initialization, acute and/or chronic changes in patient physiology, disease conditions, device parameters, and/or other factors may cause a shift in the timing of signal features used for pacing response classification. The initial timing parameters for pacing response classification may be adapted from time to time to accommodate these changes. In some cases, an initialization may be performed before each capture threshold test to establish the temporal framework for pacing response classification. In some cases, after a first initialization, the process may include checking to determine if the timings of the features of interest have shifted, and if so the timing parameters of the temporal framework may be adjusted to accommodate the feature timing shifts. The adjustments may or may not require capture of a multi-sample EGM signal and could be based on a few selected features of the cardiac signal rather than the EGM. In some implementations, the current timing of signal features may be compared to the initial timing parameters to determine if a shift in the timing of the signal features has occurred. If a shift in the timing of signal features has occurred, the process may involve adjusting the temporal framework based on the timing shift. Alternatively, the system may re-initialize the temporal framework by acquiring and using an EGM signal. The determination to re-initialize the temporal framework may be based on an offset period between the initial feature timings and the current feature timings being larger than a preset threshold. In some cases, re-initialization of the temporal framework only occurs when persistent loss of capture or fusion is detected.

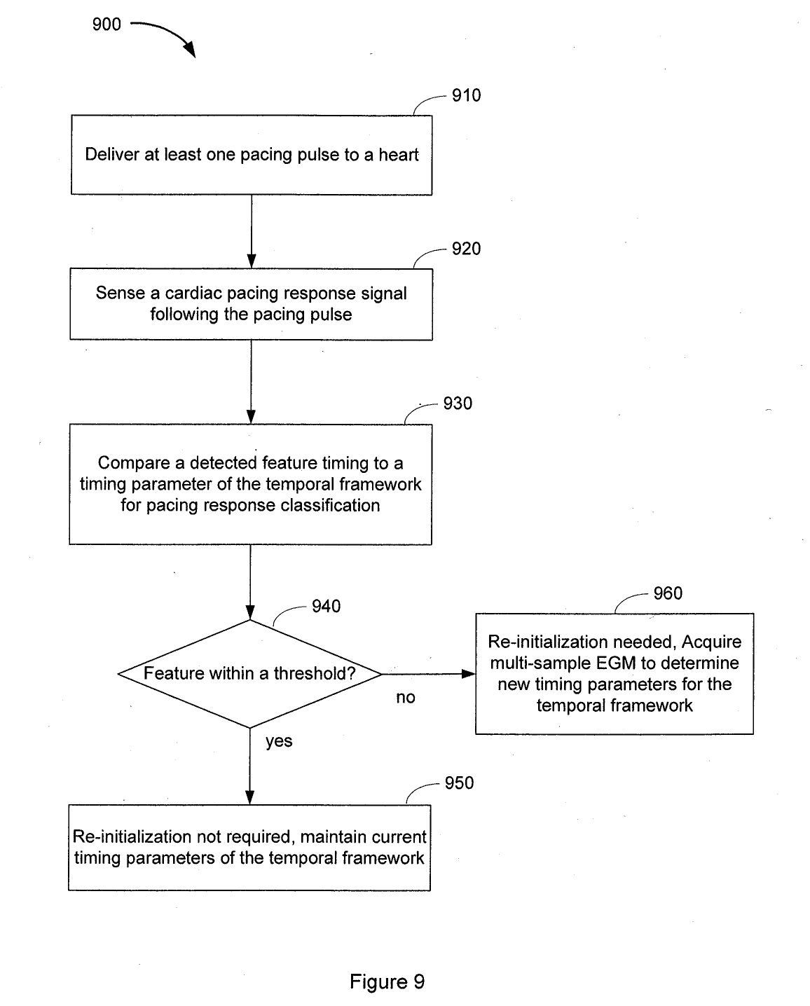

Turning now to FIG. 9, a method 900 is presented for determining if the present timings of the signal features of interest have shifted from their initial timings so as to make the initial temporal framework less effective for pacing response classification. Method 900 includes delivering 910 at least one pacing pulse to a heart. A cardiac pacing response signal is sensed 920 following the pacing pulse. Timings of one or more features of interest of the pacing response signal are detected and are compared 930 to timing parameters of the temporal framework. The process checks 940 to determine whether the current feature timing exceeds a threshold interval of an initialization timing parameter of the temporal framework. If the current feature timing is within a threshold interval from a boundary of the temporal framework, e.g., a boundary may be a start or end time of a classification interval or blanking period, then the temporal framework may be re-initialized to reset one or more of the temporal framework timing parameters. If, however, the current feature timing falls outside the predetermined time threshold, in this example, re-initialization is not required and the current timing parameters of the temporal framework for pacing response classification are maintained. Re-initialization may involve, for example, capturing the multi-sample EGM of the response signal to determine new timing parameters for the temporal framework as discussed herein.

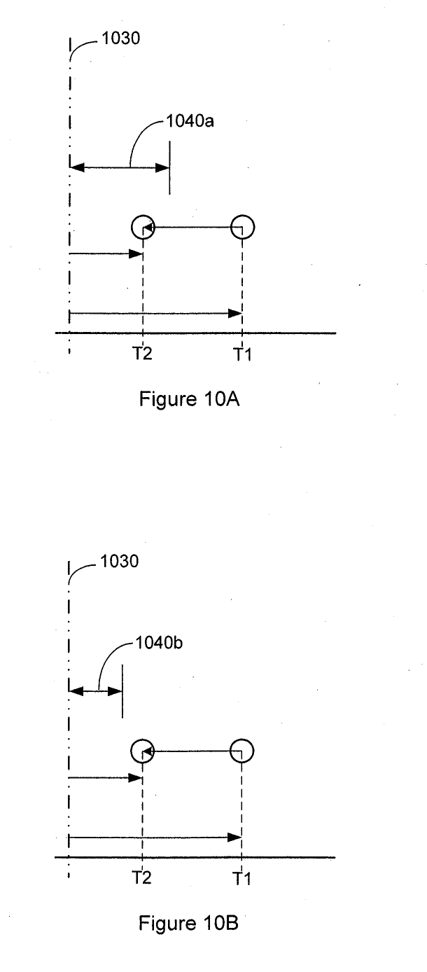

To illustrate, FIG. 10A shows a feature that initially occurs at time T1 measured from a temporal framework boundary 1030, e.g., the end time of a blanking period. Later, the feature moves to occur at time T2 which is second distance from the temporal framework boundary 1030. If the movement of the feature causes the feature to occur within a threshold interval 1040a from the temporal framework boundary 1030, then the movement triggers a re-initialization. FIG. 10B shows an example of a scenario wherein the feature has moved but falls outside the threshold interval 1040b. In this scenario, a re-initialization of the temporal framework would not be needed.

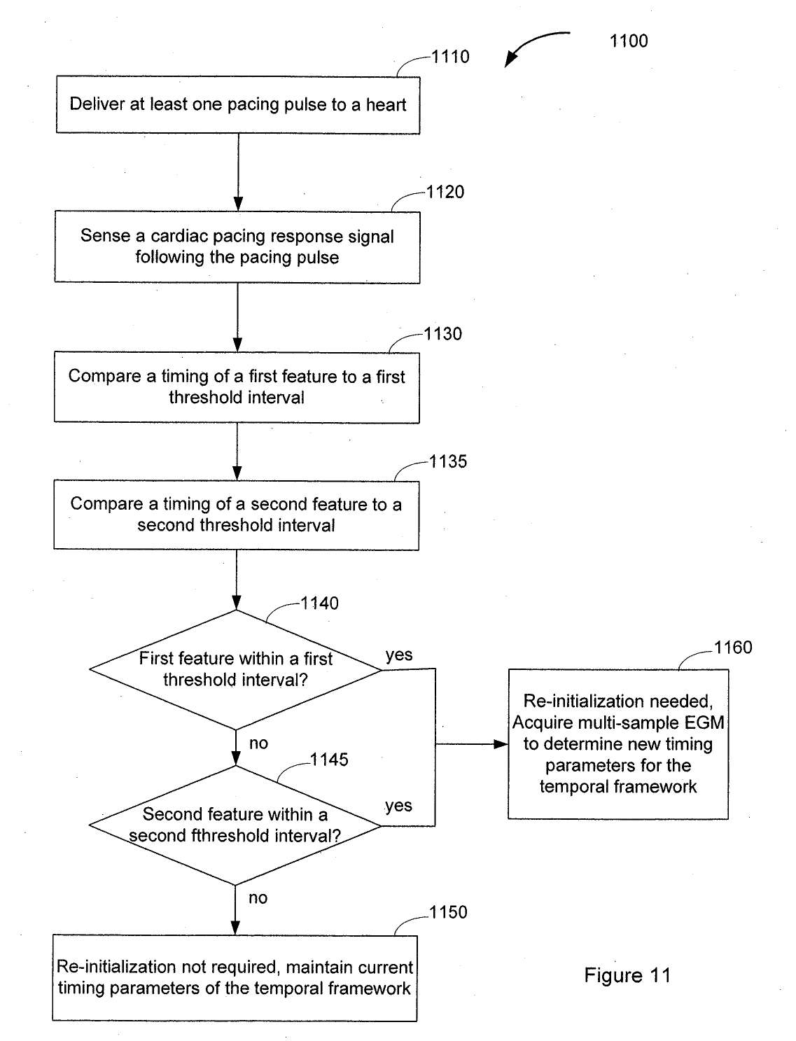

FIG. 11 is a flow diagram illustrating method 1100 that is similar to FIG. 9 in some respects with the addition of a second feature and its respective second feature threshold. As described in FIG. 11, at least one pacing pulse is delivered 1110 to a heart and a pacing response signal following the pacing pulse is sensed 1120. At least two features of the pacing response signal are detected. The timing of the first feature is compared 1130 to a timing parameter of the temporal framework. For example, the first feature timing may be compared to a first threshold interval measured from a first boundary of the temporal framework, such as the start time of the first classification interval. The timing of the second feature timing may be compared 1135 to a second threshold interval measured from a second boundary of the temporal framework, such as the start time of the second classification interval. If either the first feature timing or the second feature timing falls within 1140, 1145 the first or second threshold intervals, respectively, then the temporal framework timing parameters are re-initialized 1160. If, however, both the first feature timing and the second feature timing fall outside 1140, 1145 the threshold intervals, then re-initialization is not needed 1150 and the previous timing parameters of the temporal framework are maintained. If re-initialization is needed the multi-sample EGM may be determined 1160 to establish new timing parameters for the temporal framework. In some cases, a feature timing may be compared to more than one timing parameter of the temporal framework. For example, a feature timing may be compared to both the start time of a first classification interval and an end time of the first classification interval to determine if re-initialization is needed.

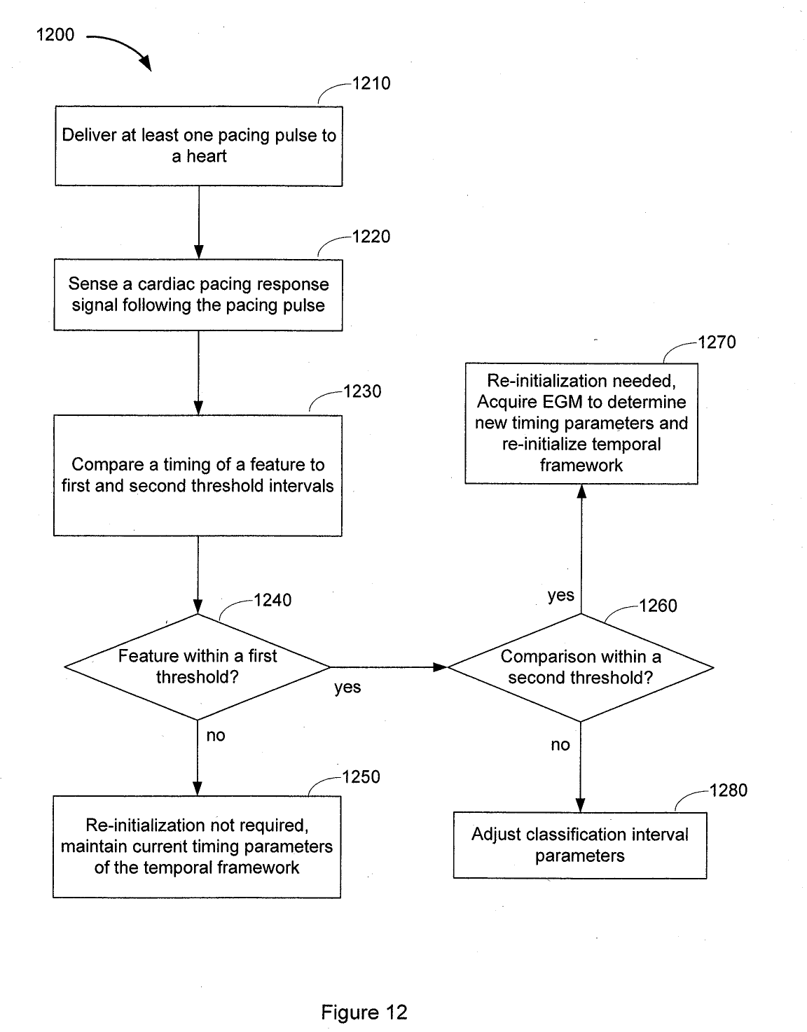

FIG. 12 is a flow diagram illustrating method 1200 that is similar to FIG. 9 in some respects with additional functionality to perform incremental adjustments of the temporal framework without acquiring an EGM signal. This process could be particularly useful to adjust the temporal framework without the computational burden of acquiring an EGM for re-initialization. Method 1200 includes delivering 1210 at least one pacing pulse to a heart and sensing 1220 the pacing response signal. Timing of a feature of the sensed cardiac response signal is compared 1230 to first and second threshold intervals measured from a temporal framework boundary, e.g., the start time of the first classification interval. In this example, the first threshold interval is greater than the second threshold interval. If the timing of the feature does not fall 1240 within the first threshold interval, re-initialization of the temporal framework timing parameters is not required 1250 and the previous timing parameters are maintained. If the feature timing falls 1240 within the first threshold interval and does not fall 1260 within the second threshold interval, the timing of the temporal framework boundary may be incrementally adjusted 1280 without acquiring an EGM signal. For example, the timing of the temporal framework boundary may be adjusted so that the feature timing falls outside the first threshold interval. If the feature timing falls 1260 within the first and second threshold intervals, re-initialization including acquisition of an EGM signal is performed 1270.

Cardiac pacing response classification may be implemented during capture threshold testing and/or non-capture threshold test therapeutic pacing for biventricular pacing. Biventricular pacing has been shown to improve the outcomes for people who suffer from congestive heart failure. Effective biventricular pacing depends on consistent capture by pacing pulses applied to both the right and left ventricles.

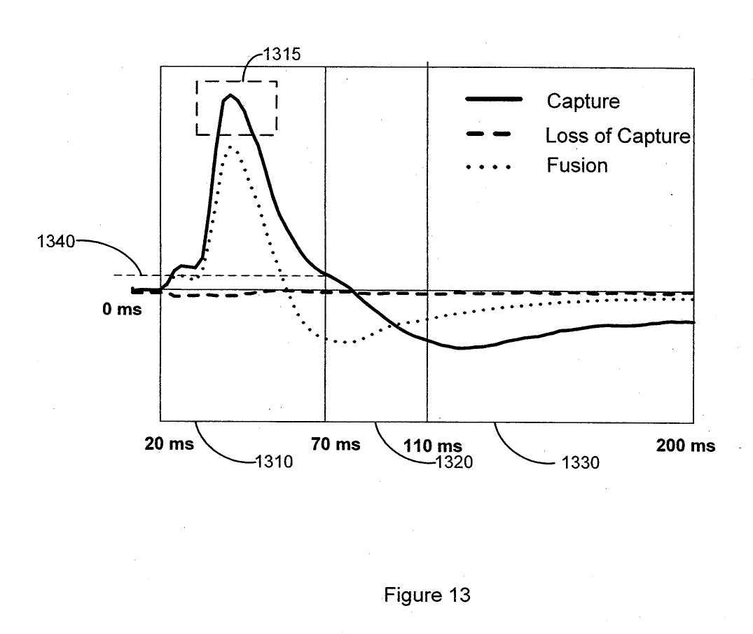

FIG. 13 illustrates a temporal framework for left ventricular pacing response classification that includes three classification intervals. The temporal framework illustrated in FIG. 13 can be used to discriminate between non-capture, capture, and fusion responses and is particularly useful for detecting these responses to left ventricular pacing. Although this example describes left ventricular pacing response determination, a similar temporal framework may be used to determine the pacing response of other heart chambers. In this example, the first classification interval 1310 and the third classification interval 1330 are left ventricular capture detection intervals and the second classification interval 1320 is a left ventricular fusion detection interval that is interposed between the first and third capture detection intervals. The first capture detection interval may start after a left ventricular (same chamber) blanking period, which may last up to about 37.5 ms and in this example lasts for about 20 ms. After the blanking period, the first capture detection interval 1310 begins, during which the device attempts to detect a signal peak indicative of potential capture. The amplitude of the response signal detected in the first capture detection interval 1310 can be used to discriminate between non-capture and potential capture or potential fusion. For example, the pacing response signal needs to exceed a threshold 1340 in the first capture detection interval 1310 in order for the cardiac response to be classified as potential capture or potential fusion.

If the peak of the pacing response signal exceeds the threshold 1340 and is detected within a capture detection region 1315 in the first capture detection interval 1310, then the pacing response is determined to be potential capture and capture is confirmed based on the pacing response signal sensed during the second and third classification intervals 1320, 1330. In some cases, the peak of a fusion response signal may be detected in the capture detection region 1315 and, in these cases, the pacing response signal in the second and third classification intervals 1320, 1330 is used to discriminate between capture and fusion.

In the illustrated example, the second classification interval 1320 is used to confirm or detect fusion and is denoted herein as the fusion detection interval. In this example, the fusion detection interval 1320 starts at about 70 milliseconds after the pacing pulse and continues for about 40 milliseconds. The fusion detection interval 1320 may start earlier or later and may be a longer or shorter period of time than is depicted in FIG. 13 depending on pacing signal morphology. The fusion detection interval 1320 may begin immediately after the first capture detection interval 1310 or may start after a delay period that occurs after the first capture detection interval 1310 ends. Discrimination between fusion and capture may depend on the timing and/or amplitude of a first peak detected in the first capture detection interval 1310 in addition to the timing and/or amplitude of a second peak that is detected within either the fusion detection interval 1320 or the second capture detection interval 1330. For example, the first peak detected in the first capture detection interval 1310 indicates potential fusion or potential capture. If a second peak falls within the fusion detection interval 1320, fusion is confirmed. If the second peak falls within the second capture detection interval 1330, capture is confirmed.

In some implementations a capture detection region 1315 is used within the first capture detection interval 1310. The capture detection region 1315 has upper and lower time boundaries and upper and lower amplitude boundaries. If the first peak of the pacing response signal falls within the capture detection region 1315, capture is indicated but may not confirmed until the second peak of the pacing response signal is detected in the second capture detection region 1330. Confirmation of potential fusion or potential capture using the fusion detection interval 1320 and the second capture detection interval 1330 is useful because of the similar left ventricular signal morphologies for fusion and capture, which can cause the first signal peaks for these responses to have similar timings and/or amplitudes.

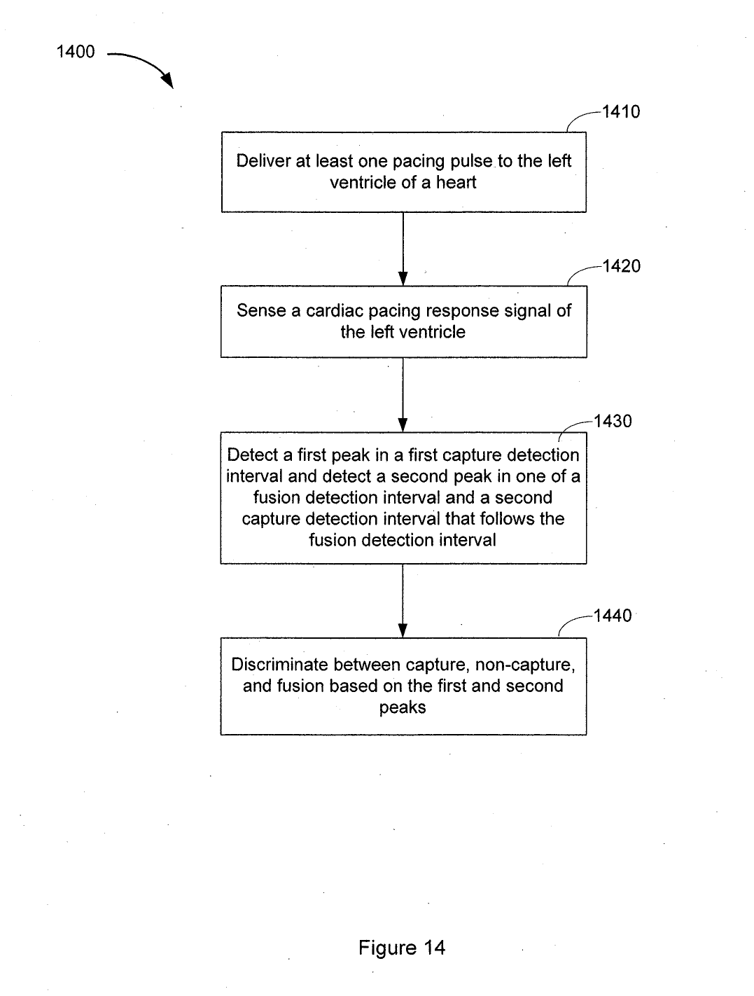

FIG. 14 is a flow diagram illustrating a method 1400 of classifying the left ventricular pacing response. The method 1400 includes delivering 1410 at least one pacing pulse to the left ventricle of the heart. The method 1400 of FIG. 14 further includes sensing 1420 a cardiac pacing response signal of the left ventricle following delivery of the pacing pulse. A first peak is detected 1430 in a first capture detection interval and a second peak is detected 1430 in one of a fusion detection interval and a second capture detection interval that follows the fusion detection interval. Method 1400 further includes discriminating 1440 between capture, non-capture, and fusion based on the first and second peaks.

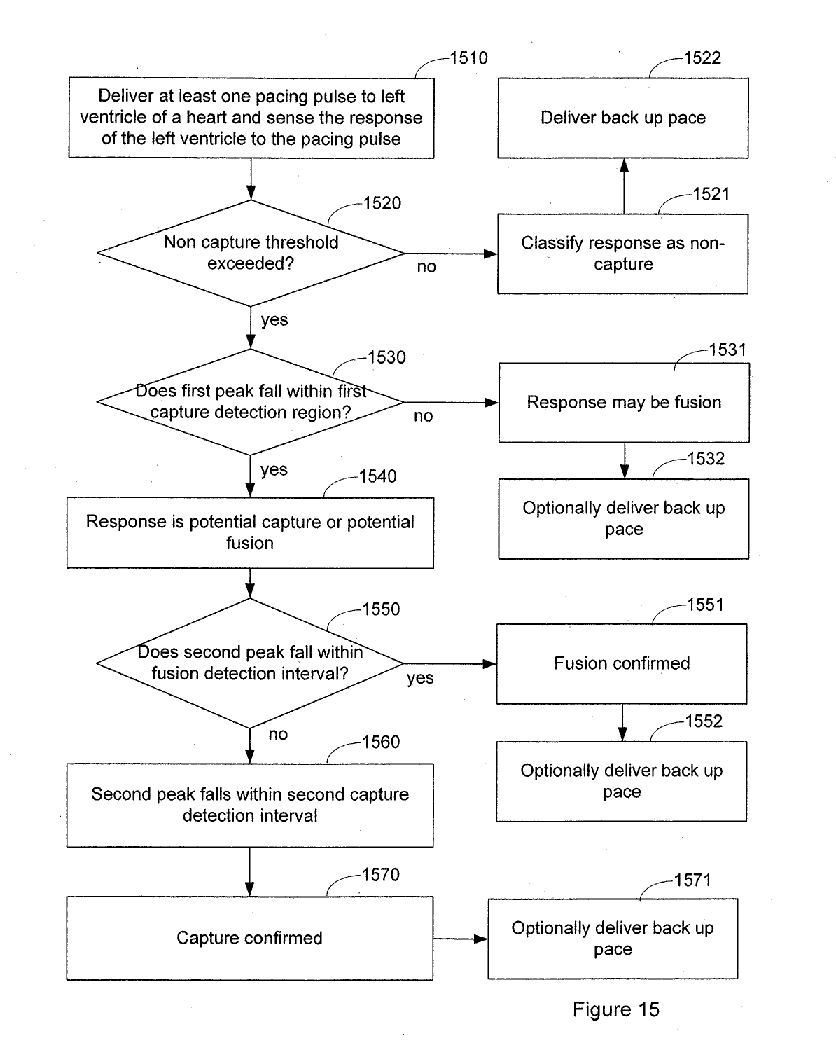

FIG. 15 is a more detailed flow diagram to describe the classification of a left ventricular pacing response. The method illustrated in FIG. 15 includes delivering 1510 at least one pacing pulse to the left ventricle of the heart and sensing 1510 the response of the left ventricle to the pacing pulse. If a non-capture threshold is not exceeded 1520, the pacing response is classified 1521 as non-capture and a back-up pace is delivered 1522. The backup pace may be delivered to the left ventricle and/or may be delivered to the right ventricle. If the non-capture threshold is exceeded 1520 and a first peak does not fall 1530 within a first capture detection interval, then the pacing response may be 1531 fusion and a back up pace is optionally delivered 1532. If the non-capture threshold is exceeded 1520 and a first peak falls 1530 within a first capture detection interval, then the response is 1540 potential capture or potential fusion. If the second peak falls 1550 within a fusion detection interval, the pacing response is confirmed 1551 to be a fusion response and a back up pace is optionally delivered 1552. If the second peak does not fall 1550 within the fusion detection interval, the second peak is determined 1560 to fall within a second capture detection interval and the pacing response is confirmed 1570 to be capture and a back-up pace is optionally delivered 1571.

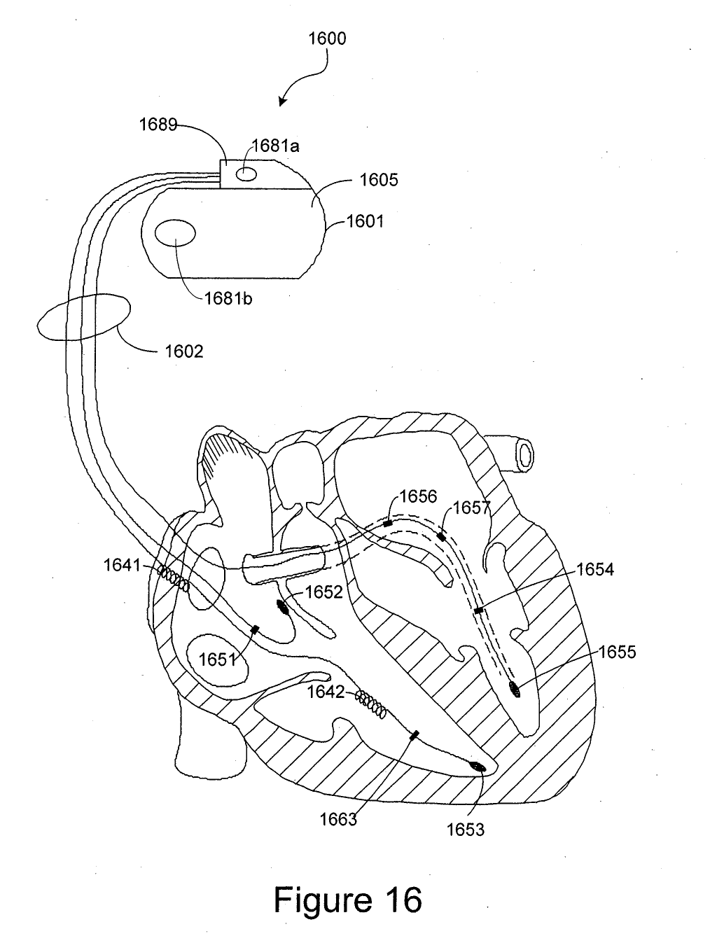

Referring now to FIG. 16 of the drawings, there is shown a cardiac rhythm management system that may be used to implement cardiac response classification methods according to embodiments described herein. The cardiac rhythm management system in FIG. 1 includes an implantable cardiac device (ICD) 1600 that is electrically and physically coupled to lead system 1602. The implantable cardiac device may include pacemakers, defibrillators, cardiac resynchronizers and/or any other type of device that delivers pacing pulses to the heart. The header and/or housing of the ICD 1600 may incorporate one or more electrodes 1681a, 1681b used to provide electrical stimulation energy to the heart and to sense cardiac electrical activity.

The lead system 1602 is used to detect electric cardiac signals produced by the heart and to provide electrical energy to the heart under certain predetermined conditions to treat cardiac arrhythmias. The lead system 1602 may include one or more electrodes used for pacing, sensing, and/or cardioversion/defibrillation. In the embodiment shown in FIG. 16, the lead system 1602 includes an intracardiac right ventricular (RV) lead system, an intracardiac right atrial (RA) lead system, and an intracardiac left ventricular (LV)/left atrial (LA) lead system. The lead system 1602 of FIG. 16 illustrates one embodiment that may be used in connection with the cardiac response classification methodologies described herein. Other arrangements may additionally or alternatively be used.

The lead system 1602 may include intracardiac leads implanted in a human body with portions of the intracardiac leads inserted into a heart. The intracardiac leads include various electrodes positionable within the heart for sensing electrical activity of the heart and for delivering electrical stimulation energy to the heart, for example, pacing pulses and/or defibrillation shocks to treat various arrhythmias of the heart.

The lead system may include one or more extracardiac leads having electrodes, e.g., epicardial electrodes, positioned at locations outside the heart for sensing and pacing one or more heart chambers.

The right ventricular lead system illustrated in FIG. 16 includes an SVC-coil 1641, an RV-coil 1642, an RV-ring electrode 1663, and an RV-tip electrode 1653. The right ventricular lead system extends through the right atrium and into the right ventricle. In particular, the RV-tip electrode 1653, RV-ring electrode 1663, and RV-coil electrode 1642 are positioned at appropriate locations within the right ventricle for sensing and delivering electrical stimulation pulses to the right ventricle. The SVC-coil 1641 is positioned at an appropriate location within the right atrium chamber or a major vein leading to the right atrial chamber. In some implementations, the lead system may not include defibrillation electrodes, e.g., shock coils 1641 and 1642.

In one configuration, the RV-tip electrode 1653 referenced to the can electrode 1681b may be used to implement unipolar pacing and/or sensing in the right ventricle. Bipolar pacing and/or sensing in the right ventricle may be implemented using the RV-tip and RV-ring electrodes 1653, 1663. In yet another configuration, the RV-ring 1663 electrode may optionally be omitted, and bipolar pacing and/or sensing may be accomplished using the RV-tip electrode 1653 and the RV-coil 1642, for example. The right ventricular lead system may be configured as an integrated bipolar pace/shock lead. The RV-coil 1642 and the SVC-coil 1641 can be used as defibrillation electrodes.

The left heart lead includes an LV distal electrode 1655 and an LV proximal electrode 1654 located at appropriate locations in or about the left ventricle for sensing signals of the left ventricle and/or delivering electrical stimulation to left ventricle. In the example of FIG. 16, the left heart lead also includes optional left atrial electrodes 1656, 1657. The left heart lead may be guided into the right atrium via the superior vena cava. From the right atrium, the left heart lead may be deployed into the coronary sinus ostium and may be guided through the coronary sinus to a coronary vein. This vein is used as an access pathway for leads to reach the surfaces of the left atrium and/or left ventricle which are not directly accessible from the right side of the heart. Some electrodes, e.g., electrodes 1656, 1657, may be used for electronic repositioning pacing. For example, electrode repositioning may involve selecting one or more electrodes from a number of possible electrodes for delivery of pacing therapy, e.g., to the LV.

Unipolar pacing and/or sensing in the left ventricle may be implemented, for example, using the LV distal electrode 1655 referenced to the can electrode 1681b. The LV distal electrode 1655 and the LV proximal electrode 1654 may be used together as bipolar sense and/or pace electrodes for the left ventricle. The electrode vector used for cardiac response classification may include, for example, any unipolar, extended bipolar and/or bipolar combination. The electrode vector used for cardiac response classification can be determined for a particular pacing vector. The left heart lead and the right heart leads, in conjunction with the ICD 1600, may be used to provide cardiac resynchronization therapy such that the ventricles and/or atria of the heart are paced substantially simultaneously, or in phased sequence, to provide enhanced cardiac pumping efficiency for patients suffering from congestive heart failure.

The right atrial lead includes a RA-tip electrode 1652 and an RA-ring electrode 1651 positioned at appropriate locations in the right atrium for sensing and pacing the right atrium. In one configuration, the RA-tip 1652 referenced to the can electrode 1681b, for example, may be used to provide unipolar pacing and/or sensing in the right atrium. In another configuration, the RA-tip electrode 1652 and the RA-ring electrode 1651 may be used to effect bipolar pacing and/or sensing.

FIG. 16 illustrates one embodiment of left atrial electrodes 1656, 1657. Unipolar pacing and/or sensing of the left atrium may be accomplished, for example, using the LA distal electrode 1657 to the can 1681b pacing vector. The LA proximal 1656 and LA distal 1657 electrodes may be used together to implement bipolar pacing and/or sensing of the left atrium.

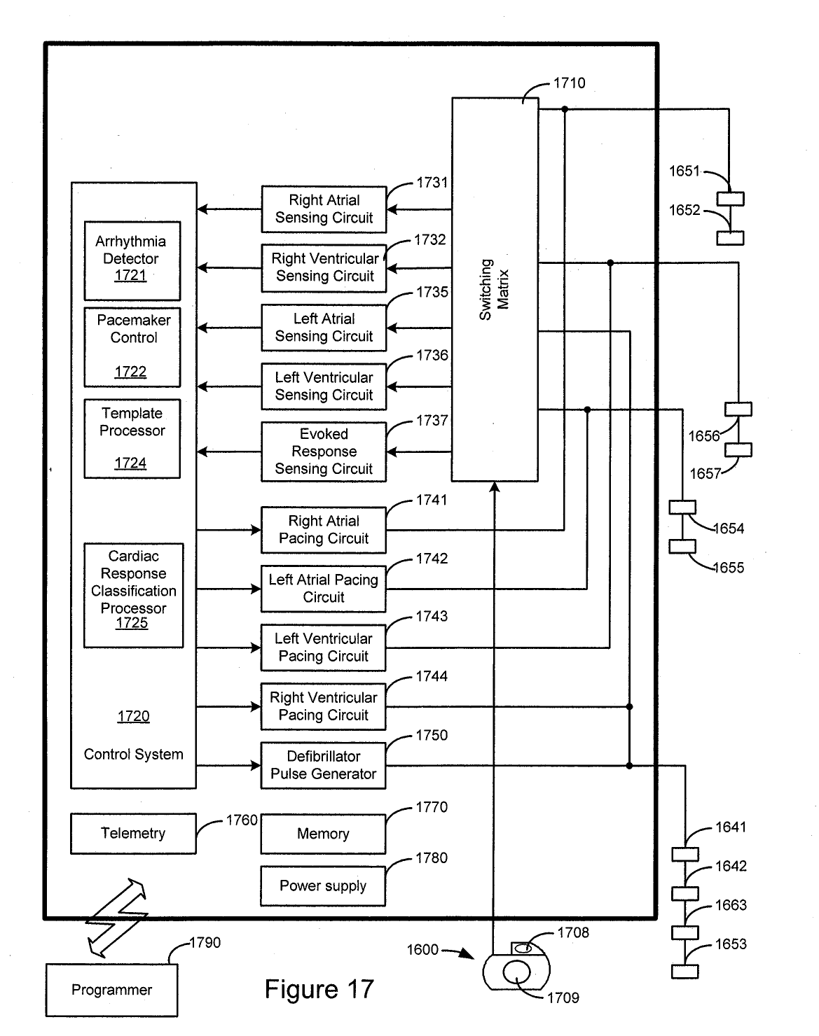

Referring now to FIG. 17, a block diagram of the circuitry of the ICD 1600 is illustrated. FIG. 17 shows the ICD divided into functional blocks. It is understood by those skilled in the art that there exist many possible configurations in which these functional blocks can be arranged. The example depicted in FIG. 17 is one possible functional arrangement. Other arrangements are also possible. For example, more, fewer or different functional blocks may be used to describe an ICD suitable for implementing the cardiac response classification methodology of the present invention. In addition, although ICD 1600 depicted in FIG. 17 contemplates the use of a programmable microprocessor-based logic circuit, other circuit implementations may be utilized.

The ICD 1600 depicted in FIG. 17 includes circuitry for receiving cardiac signals from a heart and delivering electrical stimulation energy to the heart in the form of pacing pulses or defibrillation/cardioversion shocks. In one embodiment, the circuitry of the ICD 1600 is encased and hermetically sealed in a housing suitable for implanting in a human body. Power to the ICD 1600 is supplied by an electrochemical battery 1780. A connector block (not shown) is attached to the housing of the ICD 1600 to allow for the physical and electrical attachment of the lead system conductors to the circuitry of the ICD 1600.

The ICD 1600 may be a programmable microprocessor-based system, including a control system 1720 and memory 1770. The memory 1770 may store programming instructions and/or parameters to achieve various pacing, defibrillation, and/or sensing functions. Further, the memory 1770 may store data indicative of cardiac signals received by other components of the ICD 1600. The memory 1770 may be used, for example, for storing EGM and historical therapy data. The data storage may include, for example, data obtained from long term patient monitoring used for trending or other diagnostic purposes. Historical data, as well as other information, may be transmitted to an external programmer unit 1790 as needed or desired.

The control system 1720 and memory 1770 may cooperate with other components of the ICD 1600 to control the operations of the ICD 1600. The control system 1720 depicted in FIG. 17 incorporates a cardiac response classification processor 1725 for classifying cardiac responses to pacing stimulation in accordance with various embodiments disclosed. The control system 1720 may include additional functional components including a pacemaker control circuit 1722, an arrhythmia detector 1721, and a template processor 1724 for cardiac signal morphology analysis, along with other components for controlling the operations of the ICD 1600.

Telemetry circuitry 1760 may be implemented to provide communications between the ICD 1600 and an external programmer unit 1790. In one embodiment, the telemetry circuitry 1760 and the programmer unit 1790 communicate using a wire loop antenna and a radio frequency telemetric link, as is known in the art, to receive and transmit signals and data between the programmer unit 1790 and the telemetry circuitry 1760. In this manner, programming commands and other information may be transferred to the control system 1720 of the ICD 1600 from the programmer unit 1790 during and/or after implant. In addition, stored cardiac data pertaining to timing parameters of elements within a temporal framework for pacing response classification, for example, along with other pacing response classification data, may be transferred between the programmer unit 1790 and the ICD 1600.

In the embodiment illustrated in FIG. 17, electrodes RA-tip 1652, RA-ring 1651, RV-tip 1653, RV-ring 1663, RV-coil 1642, SVC-coil 1641, LV distal electrode 1655, LV proximal electrode 1654, LA distal electrode 1657, LA proximal electrode 1656, header electrode 1708 and can electrode 1709 are coupled through a switch matrix 1710 to sensing circuits 1731-1737.

A right atrial sensing circuit 1731 serves to detect and amplify electrical signals from the right atrium. A right ventricular sensing circuit 1732 serves to detect and amplify electrical signals from the right ventricle of the heart. A left atrial sensing circuit 1735 serves to detect and amplify electrical signals from the left atrium of the heart. A left ventricular sensing circuit 1736 serves to detect and amplify electrical signals from the left ventricle of the heart. The outputs of the switching matrix 1710 may be operated to couple selected combinations of electrodes 1651, 1652, 1656, 1657, 1654, 1655, 1641, 1642, 1663, 1653 to an evoked response sensing circuit 1737. The evoked response sensing circuit 1737 may serve to sense and amplify voltages developed using various combinations of electrodes for cardiac response classification. The outputs of the sensing circuits 1731-1737 are coupled to the control system 1720.

In the embodiments described herein, various combinations of pacing and sensing electrodes may be utilized in connection with pacing and sensing the cardiac signal following the pace pulse to classify the cardiac response to the pacing pulse. For example, in some embodiments, a first electrode combination is used for pacing a heart chamber and a second electrode combination is used to sense the cardiac signal following pacing. In other embodiments, the same electrode combination is used for pacing and sensing.

Sensing the cardiac signal following a pacing pulse using the same electrode combination for both pacing and sensing may yield a sensed cardiac signal including a pacing artifact component associated with residual post pace polarization at the electrode-tissue interface. The pacing artifact component may be superimposed on a smaller signal indicative of the cardiac response to the pacing pulse, i.e., the evoked response. The pacing output circuitry may include a coupling capacitor to block DC components from the heart and to condition the pacing stimulus pulse. The presence of a large pacing artifact signal may complicate the classification of the cardiac response to pacing. In some cases, the ICD may include circuitry to cancel the pacing artifact from the detected signal. Classification of the cardiac response to pacing may be implemented using the pacing artifact cancelled signal. Cancellation of the pacing artifact in cardiac response classification is particularly important when the same or similar electrode combinations are used both for delivering pacing pulses and for sensing the cardiac signals following the delivery of the pacing pulses.

In various embodiments described herein a first electrode combination may be used for pacing the heart chamber and a second electrode combination used for sensing the cardiac signals following the pace for cardiac response classification. If different electrode combinations are used for pacing and sensing, a temporal separation between the cardiac response signal, e.g., the captured response, and the pacing artifact may facilitate classification of the cardiac response to pacing without cancellation of the pacing artifact or with reduced circuitry for canceling the pacing artifact. The temporal separation occurs due to the propagation delay of the depolarization wavefront initiated at the pacing electrode and traveling to a sensing electrode that is physically spaced apart from the pacing electrode. The temporal separation of the cardiac response signal and the pacing artifact may be sufficient to make cancellation of the pacing artifact unnecessary.

The pacemaker control circuit 1722, in combination with pacing circuitry for the left atrium, right atrium, left ventricle, and right ventricle 1742, 1741, 1743, 1744, may be implemented to selectively generate and deliver pacing pulses to the heart using various electrode combinations. The pacing electrode combinations may be used to effect bipolar or unipolar pacing of the heart chambers as described above.

Possible sensing vectors for effecting cardiac response classification may include, for example, RV-tip 1653 and RV-coil 1642, RV-coil 1642 and LV distal electrode 1655, RV coil 1642 and LV proximal electrode 1654, RV-coil 1642 and can 1681b, RV-coil 1642 and SVC coil 1641, RV-coil 1642 and SVC coil 1641 tied and the can 1681b, RV-coil 1642 and RA-ring 1651, RV-coil 1642 and RA-tip 1652, LV distal electrode 1655 and LV proximal electrode 1654, LV distal electrode 1655 and can 1681b, LV distal electrode 1655 and SVC coil 1641, LV distal electrode 1655 and RA-ring 1651, LV distal electrode 1655 and RA-tip 1652, LV proximal electrode 1654 and can 1681b, LV proximal electrode 1654 and SVC coil 1641, LV proximal electrode 1654 and RA-ring 1651, LV proximal electrode 1654 and RA-tip 156, SVC coil 1641 and can 1681b, RA-ring 1651 and can 1681b, RA-tip 1652 and can 1681b, SVC coil 1641 and RA-ring 1651, SVC coil 1641 and RA-tip 1652, RA-ring 1651 and RA-tip 1652, RA-ring 1651 and can 1681b, RA-tip 1652 and RV-coil 1642, RA-ring 1651 and RV-coil 1642, RA-tip 1652 and RV-tip 1653, RA-ring 1651 and RV-tip 1653, RV-tip 1653 and can 1681b, RV-ring 1663 and can 1681b, LV distal electrode 1655 and RV-coil 1642, LV proximal electrode 1654 and RV-coil 1642, LV distal electrode 1655 and RV-ring 1663, and LV distal electrode 1655 and RV-ring 1663. Some embodiments may include vectors that use one or more left atrial electrodes. This list is not exhaustive and other sensing vector combinations may be developed to implement cardiac response classification in accordance with embodiments of the invention. For example, other vectors may include a coronary sinus electrode, an indifferent electrode, a leadless ECG electrode, cardiac epicardial electrodes, subcutaneous electrodes, and/or other electrodes.

It is understood that the components and functionality depicted in the figures and described herein can be implemented in hardware, software, or a combination of hardware and software. It is further understood that the components and functionality depicted as separate or discrete blocks/elements in the figures in general can be implemented in combination with other components and functionality, and that the depiction of such components and functionality in individual or integral form is for purposes of clarity of explanation, and not of limitation.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A method of operating a cardiac device, comprising:
delivering a pacing pulse to a heart chamber during a cardiac cycle;
sensing a cardiac pacing response signal of the heart chamber during the cardiac cycle and following the pacing pulse in one or both of a first classification interval and a second classification interval, each of the first and second classification intervals associated with one or more timing parameters including at least a start time;
adapting at least one timing parameter of one or more of the first classification interval, the second classification interval, and one or more blanking periods based on timing of at least one signal feature of the pacing response signal and a temporal relationship between the first classification interval and the second classification interval;
applying the first and second classification intervals having the adapted timing parameters to a subsequent pacing response signal sensed following a subsequent pacing pulse delivered to the heart chamber;
determining if the signal feature of the subsequent pacing response signal falls within the first or second classification intervals that have the adapted timing parameters;
classifying a pacing response of the heart chamber to the subsequent cardiac pacing pulse based on a determination that the signal feature falls within the first or second classification intervals having the adapted timing parameters; and
delivering cardiac therapy based on classification of the pacing response.

2. The method of claim 1, wherein the signal feature comprises a positive or negative peak.

3. The method of claim 1, wherein adapting the timing parameters based on the temporal relationship of the first and second classification intervals comprises adapting a start time of the second classification interval based on an end time of the first classification interval.

4. The method of claim 1, wherein adapting the timing parameter comprises adapting a timing parameter of a blanking period based on a temporal relationship between the blanking period and one or both of the first classification interval and the second classification interval.

5. The method of claim 1, wherein adapting the timing parameter comprises adapting timing parameters of three classification intervals, the first classification interval used to detect possible capture or fusion, the second classification interval used to confirm fusion, and a third classification interval used to confirm capture.

6. The method of claim 1, wherein adapting the timing parameter comprises at least one of:
shortening one or more of the blanking periods and lengthening one or more of the first and second classification intervals; and
lengthening one or more of the blanking periods and shortening one or more of the first and second classification intervals.

7. The method of claim 1, further comprising:
comparing an amount of change in the timing of the signal feature compared to an initial timing of the signal feature to a threshold; and
determining whether to adapt one or more of the first classification interval, the second classification interval, and the one or more blanking periods based on the comparison of the amount of change.

8. The method of claim 1, further comprising:
comparing an amount of change in the timing of the signal feature compared to an initial timing of the signal feature to a threshold; and
determining whether to re-initialize timing parameters of the first classification interval, the second classification interval, and the one or more blanking periods, wherein re-initializing the timing parameters involves acquiring a multi-sample electrogram of a pacing response signal.

9. A cardiac device, comprising:
pacing circuitry configured to deliver a pacing pulse to a heart chamber during a cardiac cycle;
sensing circuitry configured to sense a cardiac pacing response signal of the heart chamber during the cardiac cycle and following the pacing pulse in one or both of a first classification interval and a second classification interval, each of the first and second classification intervals associated with one or more timing parameters including at least a start time;
control circuitry configured to
adapt at least one timing parameter of one or more of the first classification interval, the second classification interval, and one or more blanking periods based on timing of at least one signal feature of the pacing response signal and a temporal relationship between the first classification interval and the second classification interval;
apply the first and second classification intervals having the adapted timing parameters to a subsequent pacing response signal sensed following a subsequent pacing pulse delivered to the heart chamber;
determine if the signal feature of the subsequent pacing response signal falls within the first or second classification intervals that have the adapted timing parameters;
classify a pacing response of the heart chamber to the subsequent cardiac pacing pulse based on a determination that the signal feature falls within the first or second classification intervals having the adapted timing parameters; and
deliver cardiac therapy based on classification of the pacing response.

10. The device of claim 9, wherein the control circuitry is further configured to adapt the timing parameters based on the temporal relationship of the first and second classification intervals comprises adapting a start time of the second classification interval based on an end time of the first classification interval.

11. The device of claim 9, wherein the control circuitry is further configured to adapt a timing parameter of a blanking period based on a temporal relationship between the blanking period and one or both of the first classification interval and the second classification interval.

12. The device of claim 9, wherein the control circuitry is further configured to adapt timing parameters of three classification intervals, the first classification interval used to detect possible capture or fusion, the second classification interval used to confirm fusion, and a third classification interval used to confirm capture.

13. The device of claim 9, wherein the control circuitry is further configured to:
compare an amount of change in the timing of the signal feature compared to an initial timing of the signal feature to a threshold; and
determine whether to adapt one or both of the first and second classification intervals based on the comparison of the amount of change.

14. A method of operating a cardiac device, comprising:
delivering at least one pacing pulse to a heart chamber;
sensing a pacing response signal of the heart chamber following the pacing pulse;
detecting a temporal event of the pacing response signal, the temporal event comprising a point in time that falls between a first feature and a second feature of the pacing response signal; and
initializing timing parameters of one or more of pacing response classification intervals and one or more blanking periods based on the detected temporal event so that the first feature falls within a first classification interval and the second feature falls within a second classification interval.

15. The method of claim 14, wherein sensing the pacing response signal further comprises acquiring a multi-sample electrogram of the pacing response signal.

16. The method of claim 14, wherein detecting the temporal event comprises detecting a zero crossing point of the pacing response signal.

17. The method of claim 14, wherein detecting the temporal event comprises detecting an inflection point of the pacing response signal.

18. The method of claim 14, wherein detecting the temporal event comprises detecting a midpoint between a time of occurrence of the first feature and a time of occurrence of the second feature.

19. The method of claim 14, wherein:
detecting the temporal event comprises detecting a zero crossing point, an inflection point or a mid-point of the cardiac signal; and
initializing the timing parameters of the one or more classification intervals and the one or more blanking periods comprises setting a start time of a blanking period and an end time of a classification interval to coincide with the zero crossing point, inflection point or mid-point.

20. The method of claim 14, wherein initializing the timing parameters of the one or more blanking periods comprises initializing to allow sensing of the first or second feature.

21. The method of claim 14, wherein initializing the timing parameters of the one or more blanking periods comprises initializing to prevent sensing of signal features other than the first or the second features.

22. A cardiac device, comprising:
pacing circuitry configured to deliver at least one pacing pulse to a heart chamber;
sensing circuitry configured to sense a pacing response signal of the heart chamber following the pacing pulse and detect a temporal event of the pacing response signal, the temporal event comprising a point in time that falls between a first feature and a second feature of the pacing response signal; and
control circuitry configured to initialize timing parameters of one or more of pacing response classification intervals and one or more blanking periods based on the detected temporal event so that the first feature falls within a first classification interval and the second feature falls within a second classification interval.

23. The device of claim 22, wherein the sensing circuitry is further configured to acquire a multi-sample electrogram of the pacing response signal.

24. The device of claim 22, wherein:
the sensing circuitry is further configured to detect a zero crossing point, an inflection point, or a mid-point of the cardiac signal; and
the control circuitry is further configured to initialize the timing parameters of the one or more classification intervals and the one or more blanking periods and to set a start time of a blanking period and an end time of a classification interval to coincide with the zero crossing point, inflection point, or mid-point.

25. A method, comprising:
delivering a pacing pulse to a left ventricle of a heart;
sensing a cardiac pacing response signal of the left ventricle;
detecting a first peak in a first capture detection interval and detecting a second peak in one of a fusion detection interval and a second capture detection interval that follows the fusion detection interval; and
discriminating between capture and fusion based on the first and second peaks.

26. The method of claim 25, wherein detecting the first peak in the first capture detection interval comprises detecting the first peak in a capture detection region within the first capture detection interval, the capture detection region having upper and lower timing boundaries and upper and lower amplitude boundaries.

27. The method of claim 25, wherein discriminating between capture and fusion comprises classifying the pacing response as potential capture if the first peak falls within the capture detection region and confirming capture if the second peak falls within the second capture detection interval.

28. The method of claim 25, wherein discriminating between capture and fusion comprises classifying the pacing response as fusion if the second peak falls within the fusion detection interval.

29. A device, comprising:
pacing circuitry configured to deliver a pacing pulse to a left ventricle of a heart;
sensing circuitry configured to sense a cardiac pacing response signal of the left ventricle and detect a first peak in a first capture detection interval and detecting a second peak in one of a fusion detection interval and a second capture detection interval that follows the fusion detection interval; and
control circuitry configured to discriminate between capture and fusion based on the first and second peaks.

30. The device of claim 29, wherein the sensing circuitry is configured to detect the first peak in a capture detection region within the first capture detection interval, the capture detection region having upper and lower timing boundaries and upper and lower amplitude boundaries.
